(19) United States
(12) Patent Application Publication
(10) Pub. No.: US 2021/0169902 A1
(43) Pub. Date: Jun. 10, 2021

(54) USE OF OXYGENATED CHOLESTEROL SULFATES (OCS) TO TREAT INFLAMMATORY SKIN DISEASE AND SKIN LESIONS

(71) Applicants: Virginia Commonwealth University, Richmond, VA (US); Durect Corporation, Cupertino, CA (US)

(72) Inventors: Shunlin Ren, Richmond, VA (US); WeiQi Lin, Emerald Hills, CA (US); James E. Brown, Los Gatos, CA (US); Felix Theeuwes, Los Altos Hills, CA (US); Mee Jean Kim, San Francisco, CA (US); Andrew R. Miksztal, Palo Alto, CA (US); Hongwei Wu, Cupertino, CA (US); Min L. Lee, Saratoga, CA (US); Huey-Ching Su, San Jose, CA (US); Wilma Tamraz, San Jose, CA (US)

(21) Appl. No.: 17/072,994

(22) Filed: Oct. 16, 2020

Related U.S. Application Data
(63) Continuation of application No. 16/320,400, filed on Jan. 24, 2019, now abandoned.
(60) Provisional application No. 62/470,576, filed on Mar. 13, 2017.
(60) Provisional application No. 62/370,036, filed on Aug. 2, 2016.

(51) Int. Cl. A61K 31/575; A61P 17/06; A61K 9/06; A61K 47/10; A61K 47/12; A61K 47/14

(52) U.S. Cl. CPC A61K 31/575; A61P 17/06; A61K 47/14; A61K 47/10; A61K 47/12; A61K 9/06

(57) ABSTRACT

Methods of treating and prophylactically treating inflammatory skin diseases and skin lesions are provided. For instance, the methods may involve contacting the skin with an oxygenated cholesterol sulfate (OCS), e.g. 5-cholesten-3, 25-diol, 3-sulfate (25HC3S) or a pharmaceutically acceptable salt thereof.

### FIELD OF THE INVENTION

The present disclosure generally relates to the treatment and prophylactic treatment of inflammatory skin disease and/or skin lesions.

### INTRODUCTION

There are limited effective treatments currently available for many inflammatory skin diseases, such as dermatitis (including contact dermatitis, atopic dermatitis, and eczema). Dermatitis refers to a number of skin conditions that inflame the skin and are characterized by redness, swelling, blistering, scabbing, scaling, oozing, and/or itching. Some types of dermatitis are caused by allergies, but the majority of them do not have known causes. Common irritants which are known to sometimes cause dermatitis include soaps, saliva, various foods, detergents, baby lotions, and perfumes. Plants (especially poison ivy, oak and sumac), as well as metals (e.g. nickel, chrome, and mercury), cosmetics, and certain medications can also cause contact dermatitis. One option for treating contact dermatitis is antihistamines, e.g. diphenhydramine (Benadryl®) and hydroxyzine (Atarax®). However, these medications may cause drowsiness and are not always effective. Another option is steroid creams, which help decrease skin inflammation, itching and swelling. However, the overuse of steroids can damage the skin. In addition, there are many other types of skin inflammation, e.g. UV erythema, psoriasis, and erythropoietic protoporphyria (EPP), for which treatments options are limited, with glucocorticoids and anti-TNF antibodies being the usual choices. However, many times these agents either lack effectiveness or have to be given systemically and may thus cause unwanted side effects. Psoriasis in particular is extremely difficult to control or cure.

Skin lesions are also notoriously recalcitrant to treatment, whether or not they are caused by or associated with inflammation. For example, diabetic ulcers are difficult to treat and can result in dire health consequences if they fail to heal quickly and properly.

In view of the above, there is a need for improved agents and methods to treat and prophylactically treat inflammatory skin diseases and skin lesions. For instance, there is a need for alternative methods to treat and prophylactically treat inflammatory skin diseases and skin lesions, without significant side effects.

### SUMMARY

The present disclosure addresses these needs and provides methods of treating and/or prophylactically treating inflammatory skin diseases and skin lesions by administering one or more oxygenated cholesterol sulfates (OCS) to a subject in need thereof.

Aspects of the disclosure include:

1. A method of treating or prophylactically treating an inflammatory skin disease or a skin lesion in a subject in need thereof, comprising

administering to the subject an amount of one or more oxygenated cholesterol sulfates (OCS) that is sufficient to treat or prophylactically treat the inflammatory skin disease or the skin lesion.

2. The method of aspect 1, wherein the inflammatory skin disease comprises at least one of psoriasis, dermatitis, erythropoietic protoporphyria (EPP), and ultraviolet (UV) erythema.
3. The method of aspect 1, wherein the inflammatory skin disease comprises psoriasis.
4. The method of aspect 1, wherein the inflammatory skin disease comprises dermatitis.
5. The method of aspect 4, wherein the dermatitis comprises contact dermatitis.
6. The method of aspect 4, wherein the dermatitis comprises atopic dermatitis.
7. The method of aspect 4, wherein the dermatitis comprises eczema.
8. The method of aspect 4, wherein the dermatitis comprises seborrhoeic dermatitis.
9. The method of aspect 4, wherein the dermatitis comprises xerotic dermatitis.
10. The method of aspect 4, wherein the dermatitis comprises nummular dermatitis.
11. The method of aspect 1, wherein the inflammatory skin disease comprises erythropoietic protoporphyria (EPP).
12. The method of aspect 1, wherein the inflammatory skin disease comprises ultraviolet (UV) erythema.
13. The method of aspect 1, wherein the skin lesion comprises a skin ulcer, such as a diabetic ulcer.
14. The method of aspect 13, wherein the skin ulcer comprises a neurotrophic ulcer, a venous ulcer, an arterial ulcer or an ischemic ulcer.
15. The method of aspect 14, wherein the neurotrophic ulcer comprises a diabetic ulcer.
16. The method of aspect 13, wherein the skin ulcer comprises a decubitus ulcer.
17. The method of any one of aspects 1 to 16, wherein the one or more OCS comprises 5-cholesten-3, 25-diol, 3-sulfate (25HC3S) or a pharmaceutically acceptable salt thereof.
18. The method of any one of aspects 1 to 16, wherein the one or more OCS comprises 5-cholesten-3, 25-diol, disulfate (25HCDS) or a pharmaceutically acceptable salt thereof.
19. The method of any one of aspects 1 to 18, wherein the one or more OCS is administered to the subject at a dose ranging from about 0.001 mg/kg/day to about 100 mg/kg/day.
20. The method of any one of aspects 1 to 19, wherein the one or more OCS is administered to the subject at a dose ranging from about 0.01 mg/kg/day to about 10 mg/kg/day.
21. The method of any one of aspects 1 to 20, wherein the one or more OCS is administered to the subject at a dose ranging from about 0.1 mg/kg/day to about 1 mg/kg/day.
22. The method of any one of aspects 1 to 21, wherein the one or more OCS is administered to the subject at a dose ranging from 1 μg/unit of dosing to 10 mg/unit of dosing.
23. The method of aspects 19 and 22, wherein a unit of dosing is one injection.
24. The method of aspects 19 and 22, wherein a unit of dosing is 1 mL of a cream.
25. The method of any one of aspects 1 to 24, wherein the one or more OCS is administered at a frequency ranging from daily to annually.
26. The method of any one of aspects 1 to 25, wherein the one or more OCS is administered at a frequency ranging from daily to half-yearly.
27. The method of any one of aspects 1 to 26, wherein the one or more OCS is administered at a frequency ranging from daily to quarterly.
28. The method of any one of aspects 1 to 27, wherein the one or more OCS is administered at a frequency ranging from daily to monthly.
29. The method of any one of aspects 1 to 28, wherein the one or more OCS is administered at a frequency ranging from daily to weekly.
30. The method of any one of aspects 1 to 29, wherein the administering is performed by at least one of locally and systemically.
31. The method of any one of aspects 1 to 30, wherein the administering is performed by at least one of topically, orally and by injection.
32. The method of any one of aspects 1 to 31, wherein the administering is performed topically.
33. The method of any one of aspects 1 to 32, wherein the administering is performed by injection.
34. The method of any one of aspects 1 to 33, wherein the administering is performed by daily injection.
35. The method of any one of aspects 1 to 33, wherein the administering is performed by weekly injection.
36. The method of any one of aspects 1 to 33, wherein the administering is performed by monthly injection.
37. The method of any one of aspects 1 to 36, wherein the administering is performed by intra-lesional injection.
38. The method of any one of aspects 1 to 36, wherein the administering is performed by subcutaneous injection.
39. The method of any one of aspects 1 to 36, wherein the administering is performed by intramuscular injection.
40. The method of any one of aspects 1 to 36, wherein the administering is performed by intravenous injection.
41. The method of any one of aspects 1 to 32, wherein the administering is performed orally.
42. The method of any one of aspects 1 to 41, wherein the one or more OCS is administered as a pharmaceutical formulation, wherein the pharmaceutical formulation comprises at least one pharmaceutically acceptable excipient.
43. The method of aspect 42, wherein the pharmaceutical formulation is a lotion or cream.
44. The method of aspect 42, wherein the pharmaceutical formulation is a controlled release formulation.
45. The method of aspect 42, wherein the pharmaceutical formulation is a suspension.
46. The method of any one of aspects 42 to 45, wherein the at least one pharmaceutically acceptable excipient comprises at least one oligosaccharide.
47. The method of aspect 46, wherein the at least one oligosaccharide comprises a linear oligosaccharide, a branched oligosaccharide or a cyclic oligosaccharide.
48. The method of aspect 46, wherein the at least one oligosaccharide comprises a cyclodextrin or cyclodextrin derivative.
49. The method of aspect 48, wherein the cyclodextrin or cyclodextrin derivative comprises hydroxypropyl-β-cyclodextrin.
50. The method of any one of aspects 42 to 49, wherein the at least one pharmaceutically acceptable excipient comprises at least one alcohol.
51. The method of aspect 50, wherein the at least one alcohol comprises a diol.
52. The method of any one of aspects 42 to 51, wherein the at least one pharmaceutically acceptable excipient comprises propylene glycol.
53. The method of any one of aspects 42 to 52, wherein the at least one pharmaceutically acceptable excipient comprises at least one polyalkylene glycol.
54. The method of any one of aspects 42 to 53, wherein the at least one pharmaceutically acceptable excipient comprises at least one polyethylene glycol.
55. The method of any one of aspects 42 to 54, wherein the at least one pharmaceutically acceptable excipient comprises at least one polysorbate.
56. The method of any one of aspects 42 to 55, wherein the at least one pharmaceutically acceptable excipient comprises at least one salt.
57. The method of aspect 56, wherein the at least one salt comprises sodium chloride.
58. The method of any one of aspects 42 to 57, wherein the at least one pharmaceutically acceptable excipient comprises at least one preservative.
59. The method of any one of aspects 42 to 58, wherein the at least one pharmaceutically acceptable excipient comprises at least one buffer.
60. The method of any one of aspects 42 to 59, wherein the pharmaceutical formulation comprises phosphate buffered saline.
61. The method of any one of aspects 42 to 60, wherein the pharmaceutical formulation does not comprise hydroxypropyl cyclodextrin.
62. The method of any one of aspects 42 to 61, wherein the pharmaceutical formulation does not comprise hydroxypropyl-β-cyclodextrin.
63. One or more oxygenated cholesterol sulfates (OCS) as defined in any one of aspects 1, 17 and 18 for use in a method of treating or prophylactically treating an inflammatory skin disease or a skin lesion.
64. One or more oxygenated cholesterol sulfates (OCS) for use of aspect 63, wherein the method is a method as defined in any one of aspects 1 to 62.
65. Use of one or more oxygenated cholesterol sulfates (OCS) as defined in any one of aspects 1, 17 and 18 for the manufacture of a medicament for use in a method of treating or prophylactically treating an inflammatory skin disease or a skin lesion.
66. Use of claim 65, wherein the method is a method as defined in any one of aspects 1 to 62.
Further aspects include:
67. A composition comprising:

an oxygenated cholesterol sulfate (OCS);

a skin penetration enhancer; and

a thickening agent.

68. The composition of aspect 67, wherein the OCS comprises 5-cholesten-3, 25-diol, 3-sulfate (25HC3S) or a pharmaceutically acceptable salt thereof.
69. The composition of aspect 67, wherein the OCS comprises 5-cholesten-3, 25-diol, disulfate (25HCDS) or a pharmaceutically acceptable salt thereof.
70. The composition of any one of aspects 67 to 69, wherein the OCS is present in an amount ranging from about 0.1 wt % to about 50 wt %, based on weight of the composition.
71. The composition of any one of aspects 67 to 70, wherein the OCS is present in an amount ranging from about 0.5 wt % to about 10 wt %, based on weight of the composition.
72. The composition of any one of aspects 67 to 71, wherein the skin penetration enhancer comprises at least one member selected from alkanol, fatty alcohol, fatty acid, fatty acid ester, and polyol.
73. The composition of any one of aspects 67 to 72, wherein the skin penetration enhancer comprises at least one member selected from ethanol, dimethylsulfoxide, oleyl alcohol, isopropyl alcohol, isopropyl myristate, cetyl alcohol, polysorbate, propylene glycol monolaurate, sorbitan laurate, 2-(2-ethoxyethoxy)ethanol, caprylocaproyl polyoxyl-8 glyceride, polyglycervl oleate, polyoxyethylated glycolysed glyceride, oleic acid, a cyclodextrin or cyclodextrin derivative, propylene glycol, dipropylene glycol, polyethylene glycol, PEGylated caprylic/capric glyceride, pyrrolidone, 2-pyrrolidone, N-methyl-pyrrolidone, sodium lauryl sulfate, laurocapram, and lecithin isopropyl palmitate.
74. The composition of any one of aspects 67 to 73, wherein the skin penetration enhancer comprises at least one member selected from ethanol, cetyl alcohol, polysorbate, propylene glycol monolaurate, sorbitan laurate, 2-(2-ethoxyethoxy)ethanol, caprylocaproyl polyoxyl-8 glyceride, polyglyceryl oleate, polyoxyethylated glycolysed glyceride, oleic acid, a cyclodextrin or cyclodextrin derivative, propylene glycol, dipropylene glycol, polyethylene glycol, PEGylated caprylic/capric glyceride and lecithin isopropyl palmitate.
75. The composition of any one of aspects 67 to 74, wherein the skin penetration enhancer comprises PEG-8 caprylic/capric glyceride.
76. The composition of any one of aspects 67 to 75, wherein the skin penetration enhancer comprises (2-hydroxypropyl)-beta-cyclodextrin.
77. The composition of any one of aspects 67 to 76, wherein the skin penetration enhancer is present in the composition in an amount ranging from about 1 wt % to about 98 wt %, based on weight of the composition.
78. The composition of any one of aspects 67 to 77, wherein the skin penetration enhancer is present in the composition in an amount ranging from about 5 wt % to about 50 wt %, based on weight of the composition.
79. The composition of any one of aspects 67 to 78, wherein the skin penetration enhancer is present in the composition in an amount ranging from about 7 wt % to about 20 wt %, based on weight of the composition.
80. The composition of any one of aspects 67 to 79, wherein the thickening agent comprises surfactant.
81. The composition of any one of aspects 67 to 80, wherein the thickening agent comprises non-ionic surfactant.
82. The composition of any one of aspects 67 to 81, wherein the thickening agent comprises amphiphilic surfactant.
83. The composition of any one of aspects 67 to 82, wherein the thickening agent comprises at least one member selected from polyacrylic acid, polyacrylic acid crosslinked with allyl sucrose, polyacrylic acid crosslinked with allyl pentaerythritol, polyacrylic acid and C10-C30 alkyl acrylate crosslinked with allyl pentaerythritol, poly(ethylene glycol)-block-poly(propylene glycol)-block-poly(ethylene glycol), poloxamer, cellulose derivative, methylcellulose, carboxymethylcellulose, and carbomer.
84. The composition of any one of aspects 67 to 83, wherein the thickening agent comprises a poloxamer whose poly(propylene glycol) block has a molecular weight of 1500 to 5000 g/mol and a poly(ethylene glycol) weight fraction of 70 to 90 wt %; such as poloxamer 188 and 407.
85. The composition of any one of aspects 67 to 84, wherein the thickening agent comprises a poloxamer whose poly(propylene glycol) block has a molecular weight of 1,700 to 1,900 g/mol and a poly(ethylene glycol) weight fraction of 70 to 90 wt %; preferably poloxamer 188.
86. The composition of any one of aspects 67 to 85, wherein the thickening agent is present in the composition in an amount ranging from about 0.2 wt % to about 40 wt %, based on weight of the composition.
87. The composition of any one of aspects 67 to 86, wherein the thickening agent is present in the composition in an amount ranging from about 0.2 wt % to about 2 wt %, based on weight of the composition.
88. The composition of any one of aspects 67 to 86, wherein the thickening agent is present in the composition in an amount ranging from about 10 wt % to about 40 wt %, based on weight of the composition.
89. The composition of any one of aspects 67 to 88, further comprising an emollient.
90. The composition of any one of aspects 67 to 89, further comprising at least one emollient selected from polysorbate and sorbitan laurate.
91. The composition of aspect 89 or 90, wherein the emollient is present in the composition in an amount ranging from about 2 wt % to about 10 wt %, based on weight of the composition.
92. The composition of any one of aspects 67 to 91, further comprising a pH adjuster.
93. The composition of any one of aspects 67 to 92, further comprising a pH adjuster comprising at least one member selected from trolamine, citric acid, phosphoric acid, sodium hydroxide, and monobasic sodium.
94. The composition of any one of aspects 67 to 92, further comprising a pH adjuster comprising trolamine.
95. The composition of any one of aspects 92 to 94, wherein the pH adjuster is present in the composition in an amount ranging from about 0.5 wt % to 4 wt %, based on weight of the composition.
96. The composition of any one of aspects 67 to 95, further comprising a preservative.
97. The composition of any one of aspects 67 to 96, further comprising a paraben.
98. The composition of any one of aspects 67 to 97, further comprising at least one member selected from methyl paraben, ethyl paraben, propyl paraben, and butyl paraben.
99. The composition of any one of aspects 67 to 98, further comprising a preservative comprising methyl paraben.
100. The composition of any one of aspects 96 to 99, wherein the preservative is present in the composition in an amount ranging from about 0.1 wt % to about 1 wt %, based on weight of the composition.
101. The composition of any one of aspects 67 to 100, further comprising water.
102. The composition of aspect 101, wherein the water is present in an amount ranging from about 0.5 wt % to about 90 wt %, based on weight of the composition.
103. The composition of aspect 101, wherein the water is present in an amount ranging from about 1 wt % to about 10 wt %, based on weight of the composition.
104. The composition of aspect 101, wherein the water is present in an amount ranging from about 50 wt % to about 90 wt %, based on weight of the composition.
105. The composition of any one of aspects 67 to 104, wherein the composition is not an emulsion.
106. The composition of any one of aspects 67 to 104, wherein the composition comprises a micro-emulsion.
107. The composition of any one of aspects 67 to 104, wherein the composition comprises a solution.
108. The composition of aspect 107, wherein the solution is a lotion.
109. The composition of any one of aspects 67 to 104, wherein the composition is a cream.
110. The composition of any one of aspects 67 to 104, wherein the composition comprises a gel.
111. The composition of any one of aspects 67 to 104, wherein the composition comprises a suspension.
112. The composition of any one of aspects 67 to 104, wherein the composition comprises an aerosol.
113. The composition of aspect 111, wherein the suspension comprises particles comprising the OCS.
114. The composition of aspect 113, wherein the particles have an average particle size ranging from about 1 m to about 10 μm.
115. The composition of any one of aspects 67 to 114, wherein the composition has a pH of 4 to 8, such as a pH of 4 to 7.
116. The composition of any one of aspects 67 to 115, wherein the composition has a pH of 5 to 6.
117. A composition comprising:

an oxygenated cholesterol sulfate (OCS);

a skin penetration enhancer; and

a solvent different from the skin penetration enhancer.

118. The composition of aspect 117, wherein the OCS comprises 5-cholesten-3, 25-diol, 3-sulfate (25HC3S) or a pharmaceutically acceptable salt thereof.
119. The composition of anyone of aspects 117 to 118, wherein the OCS is present in an amount ranging from about 0.1 wt % to about 50 wt %, based on weight of the composition.
120. The composition of any one of aspects 117 to 119, wherein the skin penetration enhancer comprises at least one member selected from alkanol, fatty alcohol, fatty acid, fatty acid ester, and polyol.
121. The composition of any one of aspects 117 to 120, wherein the skin penetration enhancer comprises at least one member selected from ethanol, cetyl alcohol, polysorbate, propylene glycol monolaurate, sorbitan laurate, 2-(2-ethoxyethoxy)ethanol, caprylocaproyl polyoxyl-8 glyceride, polyglyceryl oleate, polyoxyethylated glycolysed glyceride, oleic acid, a cyclodextrin or cyclodextrin derivative, propylene glycol, dipropylene glycol, polyethylene glycol, PEGylated caprylic/capric glyceride and lecithin isopropyl palmitate.
122. The composition of any one of aspects 117 to 121, wherein the skin penetration enhancer is present in the composition in an amount ranging from about 1 wt % to about 98 wt %, based on weight of the composition.
123. The composition of any one of aspects 117 to 122, wherein the solvent comprises at least one member selected from propylene carbonate, dimethylsulfoxide, polyethylene glycol, N-methyl-pyrrolidone, and mineral oil.
124. The composition of any one of aspects 117 to 123, wherein the solvent is present in the composition in an amount ranging from about 1 wt % to about 98 wt %, based on weight of the composition.
Yet further aspects include:
125. A method of treating or prophylactically treating an inflammatory skin disease or a skin lesion in a subject in need thereof, comprising

administering to the subject an amount of the composition of any one of aspects 67 to 124 that is sufficient to treat or prophylactically treat the inflammatory skin disease or the skin lesion.

126. The method of aspect 125, wherein the inflammatory skin disease comprises at least one of psoriasis, dermatitis, erythropoietic protoporphyria (EPP), and ultraviolet (UV) erythema.
127. The method of aspect 125, wherein the inflammatory skin disease comprises psoriasis.
128. The method of aspect 125, wherein the inflammatory skin disease comprises dermatitis.
129. The method of aspect 128, wherein the dermatitis comprises contact dermatitis.
130. The method of aspect 128, wherein the dermatitis comprises atopic dermatitis.
131. The method of aspect 128, wherein the dermatitis comprises eczema.
132. The method of aspect 128, wherein the dermatitis comprises seborrhoeic dermatitis.
133. The method of aspect 128, wherein the dermatitis comprises xerotic dermatitis.
134. The method of aspect 128, wherein the dermatitis comprises nummular dermatitis.
135. The method of aspect 125, wherein the inflammatory skin disease comprises erythropoietic protoporphyria (EPP).
136. The method of aspect 125, wherein the inflammatory skin disease comprises ultraviolet (UV) erythema.
137. The method of aspect 125, wherein the skin lesion comprises a skin ulcer, such as a diabetic ulcer.
138. The method of aspect 137, wherein the skin ulcer comprises a neurotrophic ulcer, a venous ulcer, an arterial ulcer or an ischemic ulcer.
139. The method of aspect 138, wherein the neurotrophic ulcer comprises a diabetic ulcer.
140. The method of aspect 137, wherein the skin ulcer comprises a decubitus ulcer.
141. The method of any one of aspects 125 to 140, wherein the one or more OCS is administered to the subject at a dose ranging from about 0.001 mg/kg/day to about 100 mg/kg/day.
142. The method of any one of aspects 125 to 141, wherein the one or more OCS is administered to the subject at a dose ranging from 1 μg/unit of dosing to 10 mg/unit of dosing.
143. The method of aspect 142, wherein a unit of dosing is 1 mL of a cream.
144. The method of any one of aspects 125 to 143, wherein the one or more OCS is administered at a frequency ranging from daily to monthly.
145. The method of any one of aspects 125 to 143, wherein the one or more OCS is administered at a frequency ranging from daily to weekly.
146. The method of any one of aspects 125 to 145, wherein the administering is performed locally.
147. The method of any one of aspects 125 to 146, wherein the administering is performed topically.
148. One or more oxygenated cholesterol sulfates (OCS) for the use of aspect 63, wherein the method is a method as defined in any one of aspects 125 to 147.
149. The method of any one of aspects 1 to 62, wherein said administering to the subject an amount of one or more oxygenated cholesterol sulfates (OCS) comprises administering to the subject a composition as defined in any one of aspects 67 to 124.
150. One or more oxygenated cholesterol sulfates (OCS) for the use of aspect 64, wherein said administering to the subject an amount of one or more oxygenated cholesterol sulfates (OCS) comprises administering to the subject a composition as defined in any one of aspects 67 to 124.
151. Use of aspect 66, wherein said administering to the subject an amount of one or more oxygenated cholesterol sulfates (OCS) comprises administering to the subject a composition as defined in any one of aspects 67 to 124.
152. The method of any one of aspects 42 to 62, wherein the pharmaceutical formulation is formulated for IV infusion and comprises dextrose and sodium chloride.
Further aspects include:
153. A composition comprising:

an oxygenated cholesterol sulfate (OCS);

hydroxypropyl β-cyclodextrin (HPbCD); and

phosphate buffered saline.

154. The composition of aspect 153, wherein the OCS is 25HC3S.
155. A composition comprising:

an oxygenated cholesterol sulfate (OCS);

polyethylene glycol 3350;

polysorbate 80;

sodium chloride; and

phosphate buffered saline.

156. The composition of aspect 155, wherein the OCS is 25HC3S.
For the avoidance of doubt, the compositions of aspects 153 to 156 can be used in methods of aspects 1 to 62, the one or more oxygenated cholesterol sulfates (OCS) for use of aspect 64 (wherein said administering to the subject an amount of one or more oxygenated cholesterol sulfates (OCS) comprises administering to the subject the said compositions) and the use of aspect 66 (wherein said administering to the subject an amount of one or more oxygenated cholesterol sulfates (OCS) comprises administering to the subject the said compositions).

Further aspects of the disclosure provide a method of treating or prophylactically treating an inflammatory skin disease or a skin lesion in a subject in need thereof, comprising administering to the subject an amount of one or more oxygenated cholesterol sulfates (OCS) that is sufficient to treat or prophylactically treat the inflammatory skin disease or the skin lesion. In some aspects, the inflammatory skin disease comprises at least one of psoriasis, dermatitis, erythropoietic protoporphyria (EPP), and ultraviolet (UV) erythema. In some aspects, the inflammatory skin disease comprises psoriasis. In some aspects, the inflammatory skin disease comprises dermatitis. In some aspects, the dermatitis comprises contact dermatitis. In some aspects, the dermatitis comprises atopic dermatitis. In some aspects, the dermatitis comprises eczema. In some aspects, the dermatitis comprises seborrhoeic dermatitis. In some aspects, the dermatitis comprises xerotic dermatitis. In some aspects, the dermatitis comprises nummular dermatitis. In some aspects, the inflammatory skin disease comprises erythropoietic protoporphyria (EPP). In some aspects, the inflammatory skin disease comprises ultraviolet (UV) erythema. In some aspects, the skin lesion comprises a skin ulcer. In some aspects, the skin ulcer comprises a neurotrophic ulcer, a venous ulcer, an arterial ulcer or an ischemic ulcer. In some aspects, the neurotrophic ulcer comprises a diabetic ulcer. In some aspects, the skin ulcer comprises a decubitus ulcer. In further aspects, the one or more OCS comprises 5-cholesten-3, 25-diol, 3-sulfate (25HC3S) or a pharmaceutically acceptable salt thereof. In yet further aspects, the one or more OCS comprises 5-cholesten-3, 25-diol, disulfate (25HCDS) or a pharmaceutically acceptable salt thereof. In yet further aspects, the one or more OCS is administered to the subject at a dose ranging from about 0.001 mg/kg/day to about 100 mg/kg/day. In yet further aspects, the one or more OCS is administered to the subject at a dose ranging from 1 g/unit of dosing to 10 mg/unit of dosing. In yet further aspects, a unit of dosing is one injection. In yet further aspects, a unit of dosing is 1 mL of a cream. In additional aspects, the one or more OCS is administered at a frequency ranging from daily to monthly. In additional aspects, the one or more OCS is administered at a frequency ranging from daily to weekly. In additional aspects, the administering is performed by at least one of locally and systemically. In additional aspects, the administering is performed by at least one of topically, orally and by injection. In additional aspects, the administering is performed topically. In additional aspects, the administering is performed by injection. In additional aspects, the administering is performed orally. In other aspects, the one or more OCS is administered as a pharmaceutical formulation, wherein the pharmaceutical formulation comprises at least one pharmaceutically acceptable excipient. In other aspects, the pharmaceutical formulation is a lotion or cream. In other aspects, the pharmaceutical formulation is a controlled release formulation. In other aspects, the pharmaceutical formulation is a suspension. In other aspects, the at least one pharmaceutically acceptable excipient comprises at least one oligosaccharide. In other aspects, the at least one oligosaccharide comprises a linear oligosaccharide, a branched oligosaccharide or a cyclic oligosaccharide. In other aspects, the at least one oligosaccharide comprises a cyclodextrin or cyclodextrin derivative. In other aspects, the cyclodextrin or cyclodextrin derivative comprises hydroxypropyl-β-cyclodextrin. In other aspects, the at least one pharmaceutically acceptable excipient comprises at least one alcohol. In other aspects, the at least one alcohol comprises a diol. In other aspects, the at least one pharmaceutically acceptable excipient comprises propylene glycol. In other aspects, the at least one pharmaceutically acceptable excipient comprises at least one polyalkylene glycol. In other aspects, the at least one pharmaceutically acceptable excipient comprises at least one polyethylene glycol. In other aspects, the at least one pharmaceutically acceptable excipient comprises at least one polysorbate. In other aspects, the at least one pharmaceutically acceptable excipient comprises at least one salt. In other aspects, the at least one salt comprises sodium chloride. In other aspects, the at least one pharmaceutically acceptable excipient comprises at least one preservative. In other aspects, the at least one pharmaceutically acceptable excipient comprises at least one buffer. In other aspects, the pharmaceutical formulation comprises phosphate buffered saline. In other aspects, the pharmaceutical formulation does not comprise hydroxypropyl cyclodextrin. In other aspects, the pharmaceutical formulation does not comprise hydroxypropyl-β-cyclodextrin.

Further aspects provide one or more oxygenated cholesterol sulfates (OCS) as defined herein for use in a method of treating or prophylactically treating an inflammatory skin disease or a skin lesion.

Additional aspects provide one or more oxygenated cholesterol sulfates (OCS) for use as described herein and for methods as described herein.

Yet further aspects provide the use of one or more oxygenated cholesterol sulfates (OCS) as defined herein for the manufacture of a medicament for use in a method of treating or prophylactically treating an inflammatory skin disease or a skin lesion. In some aspects, the method is a method as described herein.

### BRIEF DESCRIPTION OF THE DRAWINGS

The present invention is further described in the description of invention that follows, in reference to the noted plurality of non-limiting drawings, wherein:

FIGS. 1A, 1B, and C. A, incidence of histopathologic findings in injection sites of rats (males and females); B, incidence of histopathologic findings in injection sites of dogs (males and females); C, injection site swelling (total occurrences/no. of dogs).

FIG. 2. Erythema (redness) in mice treated in accordance with the examples.

FIGS. 3A and 3B. A, IL-17 and B, TNFα protein levels in psoriatic skin/lesion as measured by ELISA in accordance with the examples.

FIGS. 4A and B. Exemplary diagrams of study drug administration sites. A, Option 1; B, Option 2.

FIGS. 5A and B. Summary of LPSI Scores. A, difference between the mean drug or vehicle vs untreated LPSI scores; B, LPSI Scores of 25HC3S in Solution or Suspension Formulation: Difference between the mean drug vs vehicle LPSI scores.

FIG. 6A-C. Individual LPSI Components. Scores for A, desquamation, B, indulation and C, erythema. Difference between the mean drug vs vehicle scores, shown with 90% confidence intervals (CI).

FIG. 7. Amount of drug found in deep skin in first and second cadaver skin flux studies.

### DETAILED DESCRIPTION OF THE INVENTION

Methods for treating and/or prophylactically treating inflammatory skin diseases and skin lesions in a subject in need thereof are described herein, as are methods for treating and/or prophylactically treating conditions which lead to, cause or are caused by, or which are associated with inflammatory skin diseases. The methods generally involve contacting the affected skin, or the skin which is likely to be affected, with at least one oxygenated cholesterol sulfate (OCS), in an amount that is effective or sufficient to treat and/or prophylactically treat the disease/condition. The methods generally include identifying or diagnosing subjects who are in need of such treatment, for example, subjects who would benefit from such treatment e.g. due to being susceptible to inflammatory skin disease, or already exhibiting at least one sign or symptom of inflammatory skin disease. For example, the subject may be a member of a particular patient population such as those with skin disease resulting from acute insult (e.g. exposure or suspected exposure to a skin damaging agent), or those with chronic conditions (e.g. long-term exposure to skin-damaging agents, genetic predispositions to inflammatory skin disease, etc.) or who have other conditions (such as diabetes) that predispose them to skin disorders, and/or from other causes.

In some aspects the present disclosure provides methods in which skin inflammation is treated locally, e.g. by topical administration, by subcutaneous administration directly into or adjacent to the affected area, etc. to provide a local dose in the affected area that is sufficient to relieve symptoms. In other words, in some aspects, the present methods encompass delivery that is not systemic. However, in some aspects, routes of delivery for a particular diagnosis (such as skin inflammation or skin lesions) may be treated systemically or by more than one route of administration (e.g. systemic injection in combination with local delivery). In addition, subjects treated with a particular route as described herein (e.g. topically, or by local subcutaneous injection) may or may not also be undergoing or undergo treatment with one or more OCS administered by the same or another route for a different, comorbid disease or condition. For example, a subject may already be undergoing treatment with at least one OCS (e.g. for high cholesterol, organ failure, etc.) by taking a formulation of OCS (e.g. oral, intravenous, etc.). Such treatment does not preclude administering, in addition, a treatment for skin inflammation.

### Definitions

The following definitions are used throughout:

As used herein, “at least one” means one, two, three, four, or more.

### Treat and Prophylactically Treat

As used herein, “prophylactically treat” (“prophylactic treatment”, “prophylactically treating” etc.) and “prevent” (“prevention”, preventing” etc.) refer interchangeably to warding off or averting the occurrence of at least one symptom of an inflammatory skin disease or skin lesion, by prophylactic administration of at least one OCS to a subject in need thereof. Generally, “prophylactic” or “prophylaxis” relates to a reduction in the likelihood of the patient developing a disorder. Typically, the subject is considered by one of skill in the art to be at risk of or susceptible to developing at least one symptom of the disease or unwanted condition, or is considered to be likely to develop at least one symptom of the disease/condition in the absence of medical intervention. Generally, however, for “prevention” or “prophylactic treatment”, administration occurs before the subject has, or is known or confirmed to have, symptoms of the disease (condition, disorder, syndrome, etc.; unless otherwise indicated, these terms are used interchangeably herein). In other words, symptoms may not yet be overt or observable. The subject may be considered at risk due to a variety of factors, including but not limited to: genetic predisposition; recent certain or suspected or unavoidable future exposure to a toxic agent (e.g. a toxic chemical or medication, radiation, etc.); or exposure to or experience of another stressor or combination of stressors that is/are linked to or associated with the development of the disease/condition which is being prevented. In some aspects of the prevention of the inflammatory skin disease or skin lesion, the subject may already display symptoms of a potential precursor of inflammatory skin disease or skin lesion, for example, erythema. In such aspects, treatment of the subject prevents the noxious or harmful effects or outcomes (results) of the precursor condition. “Prevention” or “prophylactic treatment” of a disease or condition may involve completely preventing the occurrence of detectable symptoms, or, alternatively, may involve lessening or attenuating the degree, severity or duration of at least one symptom of the inflammatory skin disease that would occur in the absence of the medical interventions provided herein, i.e. unless one or more OCSs is administered. Alternatively, the subject may be experiencing early stage symptoms and what is prevented is the progression to full-blown disease.

In some aspects, the disease outcome or result that is prevented is death of the subject.

“Treat” (treatment, treating, etc.) as used herein refers to administering at least one OCS to a subject that already exhibits at least one symptom of the inflammatory skin disease or skin lesion. In other words, at least one parameter that is known to be associated with the disease has been measured, detected or observed in the subject. “Treatment” of an inflammatory skin disease or skin lesion involves the lessening or attenuation, or in some instances, the complete eradication, of at least one symptom of the inflammatory skin disease or skin lesion that was present prior to or at the time of administration of one or more OCSs. Thus, for example, treatment of psoriasis includes preventing or treating damage associated with psoriasis.

As used herein, “skin” refers to the membranous tissue forming the external covering or integument of an animal. In vertebrates, the skin comprises the epidermis and the dermis. However, the present disclosure includes preventing or treating inflammation or skin lesions of other tissues that form part of the body's barrier to the external environment, such as membranes (e.g. mucous membranes), i.e. the thin, pliable layers of tissue that line externally accessible cavities or areas of the body, such as the lining of the mouth, nose, ears, vagina, rectum, and conjunctiva of the eyes, etc.

### Diseases/Conditions to be Treated

The subjects who are treated with the compositions and methods described herein generally have been diagnosed with an “inflammatory skin disease” or an “inflammatory skin disorder” and/or are afflicted with one or more skin lesions. In some aspects, the inflammation is non-infectious inflammation, e.g. the inflammation is not associated or caused by an infectious agent. Symptoms of an inflammatory skin disease or a skin lesion may occur at a single site (location) on a subject, or may occur at multiple sites. In some aspects, one or more inflammatory skin disorders and one or more skin lesions may both occur in a subject, either at a contiguous section of skin or membrane, or at separate sites on an individual.

Inflammatory skin diseases are typically characterized by, for example, reddened, itchy, dry, rough, flaky, inflamed, and irritated skin, and the skin may also exhibit blisters, scaly plaques, etc. In some aspects, the inflammatory skin disease is acute, generally resolving within days or weeks even if untreated, and the compositions and methods of the present disclosure ameliorate symptoms during disease resolution (e.g. lessen itching, redness, etc.) and/or hasten the disappearance of symptoms. Alternatively, in some aspects, the skin inflammatory disease/disorder is chronic, e.g. without treatment, or even with conventional treatment, symptoms persist for weeks, months, or years, or even indefinitely. The use of the compositions and methods of the present disclosure ameliorate (provide relief from) symptoms of chronic skin inflammation while the disease persists (e.g. lessening itching, redness, cracking and flaking of skin, hastening the healing of skin lesions, etc.) and/or also partially or completely cure (cause the complete or nearly complete disappearance of) symptoms which would otherwise be present.

“Inflammatory skin diseases” is intended to encompass diseases and conditions caused by exposure to specific, known or identifiable etiological agents, and also diseases/conditions whose causes are less well-defined, e.g. they are due to an immune disorder or malfunction (e.g. an autoimmune reaction), to stress, to an unidentified allergy, to a genetic predisposition, etc., and/or are due to more than one factor.

A “skin lesion” as used herein refers most generally to an area of the skin that has abnormal growth or appearance compared to the skin around it. For example, the area of the skin may be one exhibiting a breach of one or more of the outer skin layers (at least the epidermis, and possibly the dermis and/or subcutis (hypodermis) which exposes underlying tissue. Skin lesions include, for example, skin ulcers i.e. a local defect, breakdown or excavation of the surface of the skin produced by sloughing of necrotic inflammatory tissue. Ulcers may be, for example, neurotrophic or ischemic in nature, including decubitus ulcers, diabetic ulcers, (which are frequently foot ulcers), etc. A decubitus ulcer, also known as a bed sore or pressure ulcer, is characterized by localized injury to the skin and/or underlying tissue usually over a bony prominence, as a result of pressure, or pressure in combination with shear. Such ulcers typically result from lying in one position so long that the circulation in the skin is compromised by the pressure, e.g. on the back or buttocks, and/or particularly over a bony prominence such as the sacrum (sacral decubitus). The compositions and methods disclosed herein may be used to treat any of the four stages (I-IV) of decubitus ulcers. The treatment of venous and arterial ulcers, typically of the leg or foot, is also encompassed. Skin lesions also include those caused by deliberate or accidental breaches, e.g. cuts, scratches, incisions, etc., with or without accompanying inflammation or infection. A skin lesion may also be referred to as a sore, open sore, etc. The underlying cause of a skin lesion may be inflammation, infection (e.g. viral or bacterial infection), neuropathy, ischemia, necrosis (e.g. as occurs in diabetic ulcers), or a combination of one or more of these. In addition, many skin diseases are caused by and/or characterized by both inflammation and one or more skin lesions, and all such skin diseases and/or lesions, or symptoms thereof, can be treated by the compositions and methods disclosed herein.

For the avoidance of doubt, skin lesion includes skin necrosis. Thus, the methods and techniques described herein are suitable for treating or prophylactically treating skin necrosis.

Inflammatory skin diseases/disorders (particularly chronic inflammatory skin diseases), include but are not limited to, for example: atopic dermatitis, all types of psoriasis, acne, ichthyosis, contact dermatitis, eczema, photodermatoses, dry skin disorders, herpes simplex, zoster (shingles), sunburn (e.g. severe sunburn), etc. References herein to psoriasis refer to all types of psoriasis unless otherwise specified.

In some aspects, the disease/condition that is treated is psoriasis, including plaque flexural, guttate, pustular, nail, photosensitive, and erythrodermic psoriasis. Psoriasis is generally recognized as an immune disorder and may be triggered by or associated with factors such as infection (e.g. strep throat or thrush), stress, injury to skin (cuts, scrapes, bug bites, severe sunburns), certain medications (including lithium, antimalarials, quinidine, indomethacin), etc. and may be comorbid with other immune conditions such as type 2 diabetes, cardiovascular disease, high blood pressure, Crohn's Disease, high cholesterol, depression, ulcerative colitis, etc. Psoriasis due to any of these causes, or any other cause or an unknown cause, may be treated by the formulations and methods described herein.

In some cases, individuals (patients) are defined as having psoriasis if they exhibit one of the following: 1) inflamed swollen skin lesions covered with silvery white scale (plaque psoriasis or psoriasis vulgaris); 2) small red dots appearing on the trunk, arms or legs (guttate psoriasis); 3) smooth inflamed lesions without scaling in the flexural surfaces of the skin (inverse psoriasis); 4) widespread reddening and exfoliation of fine scales, with or without itching and swelling (erythrodermic psoriasis); 5) blister-like lesions (pustular psoriasis); 6) elevated inflamed scalp lesions covered by silvery white scales (scalp psoriasis); 7) pitted fingernails, with or without yellowish discoloration, crumbling nails, or inflammation and detachment of the nail from the nail bed (nail psoriasis).

In some aspects, the disease/condition that is treated is a form of dermatitis, which is a general term as defined by inflammation of the skin. Dermatitis is also referred to in the art as eczema. Eczema can also be referred to as “atopic dermatitis”, e.g. see the website of the American Academy of Dermatology located at “aad.org/public/diseases/eczema/atopic-dermatitis”. These designations may be used interchangeably herein to describe a variety of conditions that cause an itchy, inflamed skin rash, and to refer to any superficial inflammatory process involving primarily the epidermis, marked early by redness, itching, minute papules and vesicles, weeping, oozing, and crusting, and later by scaling, lichenification, and often pigmentation.

Various types of atopic dermatitis/eczema are known, including asteatotic eczema, eczema herpeticum, nummular eczema, neurodermatitis, xerotic eczema erythema (dry scaling, fine cracking, and pruritus of the skin, occurring chiefly during the winter when low humidity in heated rooms causes excessive water loss from the stratum corneum), and seborrhoeic dermatitis. These conditions are generally non-contagious disorders characterized by chronically inflamed skin and sometimes intolerable itching, and are often associated with stress and allergic disorders that involve the respiratory system, such as asthma and hay fever. Although atopic dermatitis can appear at any age, it is most common in children and young adults, e.g. infantile eczema. Characterized by skin that oozes and becomes encrusted, infantile eczema most often occurs on the face and scalp. The condition usually improves before the child's second birthday, and medical attention can keep symptoms in check until that time.

The infantile form of eczema may first appear soon after birth, often by the fourth month of the infant's life. Infantile eczema is generally manifested as red, dry, slightly scaly, cracked, and excoriated skin, or sometimes moist and oozing skin. Infantile eczema is most frequently manifested around the face, scalp, neck, and diaper areas. Older children and young adults generally experience manifestation of the disease in the flexural areas and the cheeks. In fewer than half of the individuals inflicted with infantile eczema, the disease clears up by the age of four; yet even in these individuals, the disease may occur at a later age. The majority of eczema victims still experience occasional flare ups through the young adult years, up until about the age of thirty, at which time the disease usually disappears.

The adult form of eczema is generally manifested in the antecubital and popliteal areas, and in some cases around the hands, feet, and face. The infected skin is generally dry, erythematous, and excoriated with bacterial crusting and redness.

The localized form of eczema, which occurs in diverse individuals, is primarily manifested around the wrists, ankles, hands, feet and ears, as well as the perianal, perivulvar, and scrotal regions.

Among the adverse consequences of atopic eczema is the pruritis or itching which is associated with this disease. Those inflicted with atopic eczema often find pruritis to be a life-long companion. Any relief to be had from such intolerable itching is a clinical benefit to the affected subject. There are many factors which play a role in the occurrence of atopic eczema, such as dietetic and emotional factors. Moreover, seasonal fluctuations are an important factor with atopic eczema generally becoming worse during the winter season.

One of the greatest fears of those who are inflicted with atopic eczema, is the increased risk of viral infection, and in particular, to the infestation by a herpes simplex virus or a vaccinia virus. Additionally, those suffering from atopic eczema are abnormally susceptible to environmental irritants. Consequently, those afflicted with the disease are often advised to wear clothing which is soft and light; to stay away from heat sources; to take brief baths or showers not exceeding five minutes and using a minimal amount of soap; to avoid primary irritants such as paints, cleansers, solvents, chemical sprays, dusts, and the like; and sometimes to change their residence to a warm, dry temperature, unvarying climate where temperature extremes are rarely experienced.

In one aspect, the atopic dermatitis is contact allergic dermatitis, caused, for example, by exposure to an agent that causes an allergic reaction. Common triggers of atopic dermatitis include, for example, soap and household cleaners (e.g. all-purpose cleaners, dish detergents, laundry detergent, window cleaners, furniture polish, drain cleaners, toilet disinfectants, etc.); clothing (e.g. rough fabrics like wool); heat; contact with latex; cosmetics and ingredients of cosmetics (e.g. ascorbic acid, paraban preservatives, and alpha hydroxy acids such as glycolic acid, malic acid, and lactic acid); oils from plants such as poison ivy, poison oak, and poison sumac; contact with foods, especially acidic foods or spices; nickel, a common component of costume jewelry, watchbands, zippers, etc.; sunscreen and ingredients thereof, e.g. para-aminobenzoic acid (PABA)-based chemicals; etc.

In some aspects, the skin inflammation that is prevented or treated is “diaper rash”, which can occur in infants but also in other incontinent individuals. Diaper rash may be classified as i) irritant or contact dermatitis; or ii) may be due to a skin infection such as a Staphlococcal or Streptococcal bacterial infection or a yeast/fungal infection (e.g. Candida); or iii) caused by an allergic reaction, e.g. to cleaning products, diaper components, etc.

In other aspects, the skin inflammation that is prevented or treated is rosacea. The precise cause of this skin condition is unknown. Symptoms can include flushing and redness in the center of the face or even the shoulders, chest and back; visible broken blood vessels (spider veins); swollen, sensitive skin that may burn or itch; dry skin; rough, scaly skin; skin thickening with a bumpy texture; red and irritated eyes and swollen eyelids; etc. All types of rosacea may be prevented or treated using the compositions and methods described herein, including erythematotelangiectatic rosacea, papulopustular rosacea, phymatous rosacea, and ocular rosacea.

### Herpes Simplex

In some aspects, the treated patient has Herpes virus. Of all the Herpes viruses, the effects of Herpesvirus hominis are by far the most commonly experienced. Herpesvirus hominis, which is responsible for herpes simplex, has two different forms: Type I and Type II. Type I causes Herpes labialis (oral herpes) in the form of cold sores and unsightly lesions around the lips or nose. Type II causes Herpes genitalis (genital herpes) in the form of sores that appear below the waist, primarily in the genital area. The two types vary little with respect to the nature of their behavior and either one can take the other's place. Thus, Type II can cause a cold sore while Type I can also infect the genitals. Nevertheless, Type II is responsible for at least about eighty percent (80%) of genital herpes.

Both types I and II can be transmitted by sexual as well as non-sexual contact; however, genital herpes is generally transmitted through sexual intercourse. A Type I infection of the genitals or a Type II infection of the mouth can occur through oral-genital contact. A cold sore virus may be transmitted when two persons kiss or by means as simple as the use of the same towel to wipe their faces. The eyes can be infected simply by rubbing them after touching an infected area. Thus, there are a variety of ways in which herpes simplex viruses Types I and II can be transmitted. Moreover, although not the usual case, transmission of the viruses can even occur before the symptoms of herpes simplex appear or before the infected person is aware that he or she has herpes simplex.

The symptoms of herpes simplex infections include the development of a cluster of tiny bumps or blisters, sometimes preceded or accompanied by a fever or swollen lymph glands. The blisters then crust over, and the sores disappear—usually within three weeks after the first symptoms. However, the virus remains in the body for a lifetime, hibernating in such places as the salivary glands, the nerve tissue, and the lymph nodes. After recovery from the first attack, subsequent infections may occur over the next few years, until gradually the frequency of attacks diminishes. Occasionally, however, recurrences may appear over the rest of the individual's life. The reappearance of herpes infections is then often triggered by stress, fatigue, exposure to sun, trauma, fever or menstruation.

Other complications may develop in those who are afflicted with a herpes simplex virus. If a person suffering from herpes simplex touches a sore or blister and then rubs his eyes, he may develop a serious eye infection known as herpes keratitis. Thousands of Americans annually lose their sight because of this disease.

For women, genital herpes simplex carries special risks. To begin with, genital herpes simplex has been linked to cancer of the cervix. Female herpes victims are five to seven times more likely to develop cervical cancer than non-infected females. Genital herpes simplex can also cause serious birth defects. A pregnant woman with an active genital herpes simplex infection faces a fifty percent (50%) chance of passing the disease to her baby as the child passes through the birth canal. About fifty percent (50%) of the newborn infants who develop herpes simplex die of the infection; seventy-five percent (75%) of those who survive suffer from blindness or brain damage. Fortunately, if sores are found close to the time of delivery, the doctor can perform a Caesarean-section to prevent infection of the newborn as it passes through the birth canal.

Most Americans have been exposed to the herpes simplex virus; indeed, eighty percent (80%) of the American population carries the herpes simplex virus, and antibodies against the virus have been found in up to ninety-five percent (95%) of blood samples tested. Although some people never experience symptoms, (possibly because their immune systems repulse the virus so it cannot sustain its attack), about seven out of eight people who come in sexual contact with the herpes simplex virus will contract an infection. It is estimated that from thirty (30) to seventy (70) million Americans suffer occasionally from the most common form of herpes simplex infection, that of cold sores. Moreover, it is estimated that from five (5) to twenty (20) million Americans suffer from genital herpes simplex, and that each year, half a million more Americans join these ranks.

Since no known effective treatment for herpes simplex has existed, the total number of persons afflicted with herpes simplex continues to increase. Scientists have tried and rejected many different treatments for herpes such as vitamin C, zinc, ether, and ice packs.

### Compounds and Compositions

Examples of OCS that are used in the methods and compositions described herein include but are not limited to 5-cholesten-3, 25-diol, 3-sulfate (25HC3S); 5-cholesten-3, 25-diol, disulfate (25HCDS); 5-cholestene, 3, 27-diol, 3-sulfate; 5-cholestene, 3, 27-diol, 3, 27-disulfate; 5-cholestene, 3,7-diol, 3-sulfate; 5-cholestene, 3,7-diol, 3,7-disulfate; 5-cholestene, 3, 24-diol, 3-sulfate; 5-cholestene, 3, 24-diol, 3, 24-disulfate; 5-cholestene, 3-ol, 24, 25-epoxy 3-sulfate; and salts thereof, particularly pharmaceutically acceptable salts thereof. Disclosure of 25HC3S is found in, e.g., U.S. Pat. No. 8,399,441, which is incorporated herein by reference in its entirety. Disclosure of 25HCDS is found, e.g., in U.S. Published Application No. 20150072962, which is incorporated by reference in its entirety. In certain aspects, the OCS is selected from 5-cholesten-3, 25-diol, 3-sulfate (25HC3S) and 5-cholesten-3, 25-diol, disulfate (25HCDS) (either alone or in combination). In further aspects, the OCS is 5-cholesten-3, 25-diol, 3-sulfate (25HC3S). The OCSs are typically synthetic versions of OCSs that occur naturally in the body.

In one aspect, the OCS is 5-cholesten-3, 25-diol, 3-sulfate (25HC3S) of formula

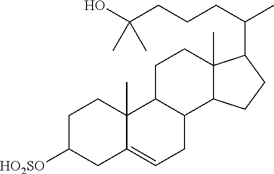

and/or a pharmaceutically acceptable salt thereof.

In one aspect, the OCS is 5-cholesten-3p, 25-diol, 3-sulfate (25HC3S) of formula

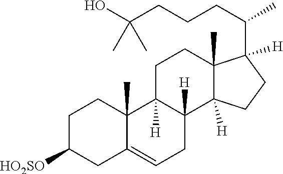

and/or a pharmaceutically acceptable salt thereof.

In one aspect, the OCS is 5-cholesten-3, 25-diol, disulfate (25HCDS) of the formula

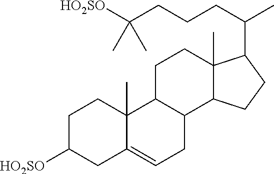

and/or a pharmaceutically acceptable salt thereof.

In some aspects, the OCS is 5-cholesten-3p, 25-diol, disulfate 25HCDS of the formula

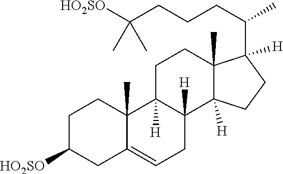

and/or a pharmaceutically acceptable salt thereof.

In some aspects, the one or more oxygenated cholesterol sulfates comprises 5-cholesten-3, 25-diol, 3-sulfate (25HC3S) or a pharmaceutically acceptable salt thereof. In some aspects, the one or more oxygenated cholesterol sulfates comprises 5-cholesten-3, 25-diol, disulfate (25HCDS) or a pharmaceutically acceptable salt thereof. In some aspects, the one or more oxygenated cholesterol sulfates consists of 5-cholesten-3, 25-diol, 3-sulfate (25HC3S) or a pharmaceutically acceptable salt thereof. In some aspects, the one or more oxygenated cholesterol sulfates consists of 5-cholesten-3, 25-diol, disulfate (25HCDS) or a pharmaceutically acceptable salt thereof.

The compounds may be administered in the pure form or in a pharmaceutically acceptable formulation (also referred to herein as a pharmaceutical formulation or a pharmaceutical composition) including suitable elixirs, binders, and the like (generally referred to as “carriers”) or as pharmaceutically acceptable salts (e.g. alkali metal salts such as sodium, potassium, calcium or lithium salts, ammonium, etc.) or other complexes. It should be understood that the pharmaceutically acceptable formulations include solid, semi-solid, and liquid materials conventionally utilized to prepare both solid, semi-solid and liquid dosage forms such as tablets, capsules, creams, lotions, and aerosolized dosage forms, etc.

Suitable pharmaceutical carriers include inert solid diluents or fillers, sterile aqueous solutions and various organic solvents. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene, isopropyl myristate or water. Other carriers/diluents include: peanut oil, ethyl cocoate, octyl cocoate, polyoxyethylenated hydrogenated castor oil, liquid paraffin, isopropanol, glycerol, propylene glycol, paraffin, celluloses, parabens, stearyl alcohol, polyethylene glycol, isopropyl myristate and phenoxyethanol. Similarly, the carrier or diluent may include any sustained release material known in the art, such as glycerol monostearate or glycerol distearate, alone or mixed with wax. In addition, the compounds may be formulated with aqueous or oil based vehicles. Water may be used as the carrier for the preparation of compositions which may also include conventional buffers and agents to render the composition isotonic.

Other potential additives and other materials (preferably those which are generally regarded as safe [GRAS]) include: colorants; flavorings; surfactants (e.g., non-ionic surfactants including polysorbate (such as TWEEN® 20, 40, 60, and 80 polyoxyethylene sorbitan monolaurate), sorbitan esters (such as Span® 20, 40, 60, and 85), and poloxamers (such as Pluronic® L44, Pluronic® F68, Pluronic® F87, Pluronic® F108 and Pluronic® F127); zwitterionic surfactant such as lecithin; anionic surfactants such as sodium dodecyl sulphate (SDS) and sulphated castor oil; and cationic surfactants such as benzalkonicum chloride and cetrimide). Surfactants include but are not limited to polyoxyl 35 castor oil (Cremophor® EL), polyoxyl 40 hydrogenated castor oil (Cremophor® RH 40), polyoxyl 60 hydrogenated castor oil (Cremophor® RH 60), d-α-tocopheryl polyethylene glycol 1000 succinate (TPGS), poly-oxyethylene esters of 12-hydroxystearic acid (e.g., Solutol® HS-15), PEG caprylic/capric glycerides, such as PEG 300 caprylic/capric glycerides (e.g., Softigen® 767), PEG caprylic/capric triglycerides, such as PEG 400 caprylic/capric triglycerides (e.g., Labrafil® M-1944CS), PEG linoleic glycerides, such as PEG 300 linoleic glycerides (e.g., Labrafil® M-2125CS), polyoxyl 8 stearate (e.g., PEG 400 monostearate), polyoxyl 40 stearate (e.g., PEG 1750 monostearate), peppermint oil, oleic acid, steric acid, etc.); and solvents, stabilizers, elixirs, and binders or encapsulants (lactose, liposomes, etc).

Solid diluents and excipients include lactose, starch, conventional disintegrating agents, coatings and the like. Preservatives such as benzyl alcohol, phenol, chlorobutanol, 2-ethoxyethanol, methyl paraben, ethyl paraben, propyl paraben, benzoic acid, sorbic acid, potassium sorbate, chlorhexidine, 3-cresol, thimerasol, phenylmercurate salts, sodium benzoate, cetrimonium bromide, benzethonium chloride, ethylhexylglycerine, alkyltrimethylammonium bromide, cetyl alcohol, steryl alcohol, chloroactamide, trichlorocarban, bronopol, 4-chlorocresol, 4-chloroxylenol, hexachloropherene, dichlorophene, or benzalkium chloride may also be used. Diluents or carriers that assist the transport of the active ingredient across the skin barrier, e.g. that are capable of crossing the keratinous layer of the skin, may be included, e.g. dimethylsulfoxide or acetic acid; as may absorption promoters such as dimethylacetamide, trichloroethanol or trifluoroethanol, certain alcohols (isopropanol, glycerol, etc.).

In some aspects of the pharmaceutical formulation, the at least one pharmaceutically acceptable excipient comprises an oligosaccharide, for example a linear oligosaccharide, a branched oligosaccharide or a cyclic oligosaccharide. The cyclic oligosaccharide may be a cyclodextrin, for example hydroxypropyl-β-cyclodextrin. In a further aspect the at least one pharmaceutically acceptable excipient does not include hydroxypropyl cyclodextrin. In a further aspect, the at least one pharmaceutically acceptable excipient does not include hydroxypropyl-β-cyclodextrin.

An oligosaccharide is a saccharide polymer containing two or more sugar molecules (monomers), for example 2 to 200 sugar molecules such as 3 to 100 sugar molecules or 3 to 10 sugar molecules. “Cyclic oligosaccharide” refers to an oligosaccharide that is cyclic. Typically a cyclic oligosaccharide comprises 5 or more sugar molecules that together form a ring, for example 5 to 200 sugar molecules such as 5 to 100 sugar molecules or 5 to 10 sugar molecules. Cyclic oligosaccharides include salts of cyclic oligosaccharides.

“Cyclodextrin” (“CD”) refers to a family of synthetic compounds comprising sugar molecules bound together in a ring (cyclic oligosaccharides). Cyclodextrins are cyclic oligosaccharides with hydroxyl groups on the outer surface and a void cavity in the center. Their outer surface is hydrophilic, and therefore they are usually soluble in water, but the cavity has a lipophilic character. The most common cyclodextrins are α-cyclodextrin, β-cyclodextrin and γ-cyclodextrin, consisting of 6, 7, and 8 α-1,4-linked glucose units, respectively. The number of these units determines the size of the cavity. Cyclodextrins typically comprise 5 or more α-D-glucopyranoside units linked 1→4, as in amylose. Typical cyclodextrins contain from six to eight units in a ring, creating a cone shape and include: a (alpha)-cyclodextrin, a 6-membered ring; β (beta)-cyclodextrin: a 7-membered ring, and γ (gamma)-cyclodextrin, an 8-membered ring. Much larger cyclodextrin rings are also known, e.g. comprising over 100 α-D-glucopyranoside units. Cyclodextrins suitable for medical purposes are readily commercially available. Cyclodextrins include salts of cyclodextrins.

Various derivatives of CDs may also be employed, including but not limited to: chloramphenicol/methyl-β-CD; highly water-soluble, randomly substituted hydroxyalkyl derivatives of β- and γ-CD such as 2-hydroxypropyl-β-cyclodextrin and 2-hydroxypropyl-γ-cyclodextrin; sulfoalkyl ether CDs such as sulfobutylether β-cyclodextrin; lipid substituted CDs; dimethyl-β-CD, randomly methylated β-CD, and the like. In some aspects, the cyclodextrin is β-cyclodextrin or sulfobutyl ether β-cyclodextrin.

Common cyclodextrin derivatives are formed by alkylation (e.g., methyl- and ethyl-β-cyclodextrin) or hydroxyalkylation of the hydroxyl groups (e.g., hydroxypropyl- and hydroxyethyl-derivatives of α-, β-, and γ-cyclodextrin) or by substituting the primary hydroxyl groups with saccharides (e.g. glucosyl- and maltosyl-β-cyclodextrin). For instance, cyclodextrin derivatives include cyclodextrins that are alkyl substituted, hydroxyalkyl substituted, sulfoalkyl ether substituted, or alkyl ether substituted, such as those in which the alkyl group comprises 1 to 8 carbons, such as 2 to 5 carbons. In such a derivative, the cyclodextrin may be fully or partially alkyl substituted, hydroxyalkyl substituted, sulfoalkyl ether substituted, or alkyl ether substituted (i.e. all or, more typically, only some of the native hydroxyl groups of the cyclodextrin are replaced with alkyl substituents, hydroxyalkyl substituents, sulfoalkyl ether substituents, or alkyl ether substituents). Cyclodextrin derivatives also include cyclodextrin ethers. Hydroxypropyl-β-cyclodextrin and its preparation by propylene oxide addition to β-cyclodextrin, and hydroxyethyl β-cyclodextrin and its preparation by ethylene oxide addition to β-cyclodextrin, were described in a patent of Gramera et al. (U.S. Pat. No. 3,459,731, issued August 1969) over 20 years ago, which is incorporated by reference herein. For a comprehensive review of cyclodextrins see Cyclodextrins and their industrial uses, editor Dominique Duchene. Editions Sante, Paris, 1987, which is incorporated by reference herein. For a more recent overview, see J. Szejtli: Cyclodextrins in drug formulations: Part 1, Pharm. Techn. Int. 3(2), 15-22 (1991); and J. Szejtli: Cyclodextrins in drug formulations: Part II, Pharm. Techn. Int. 3(3), 16-24 (1991), which is incorporated by reference herein.

Cyclodextrins approved for parenteral applications include two β-cyclodextrins (hydroxypropyl β-cyclodextrin “HPbCD”, also known as hydroxypropyl betadex, and sulfobutyl ether β-cyclodextrin “SBECD”), α-cyclodextrin and γ-cyclodextrin. HPbCD and other cyclodextrins are also approved for oral, topical, dermal, sublingual, buccal, eye drops, and nasal routes.

In some aspects of the pharmaceutical formulation, the at least one pharmaceutically acceptable excipient comprises an alcohol, for example a diol (e.g. propylene glycol). In further aspects the at least one pharmaceutically acceptable excipient comprises polyethylene glycol and/or polysorbate and/or a salt (e.g. sodium chloride) and/or a preservative and/or a buffer (e.g. phosphate buffered saline).

In some aspects of the pharmaceutical formulation, the at least one pharmaceutically acceptable excipient comprises at least one and, in some aspects, both of polyethylene glycol and polysorbate, together with, for example, phosphate buffered saline. In some aspects, such a formulation is a suspension.

In some aspects, the at least one OCS is administered as a composition that is prepared in solid forms such as tablets, pills, powders, suppositories, various slow- or extended-release formulations, and the like, or as liquid solutions, suspensions, emulsions, etc. or liquids suitable for injection and/or intravenous administration. Solid forms suitable for solution in, or suspension in, liquids prior to administration may also be prepared. The active ingredients may be mixed with excipients which are pharmaceutically acceptable and compatible with the active ingredients, e.g. pharmaceutically and physiologically acceptable carriers. Suitable excipients include, for example, water, saline, dextrose, glycerol, ethanol and the like, or combinations thereof. In addition, the composition may contain minor amounts of auxiliary substances such as wetting or emulsifying agents, pH buffering agents, and the like. Oral dosage forms may include various thickeners, flavorings, diluents, emulsifiers, dispersing aids, binders, coatings and the like. The composition of the present disclosure may contain any such additional ingredients so as to provide the composition in a form suitable for the intended route of administration. Still other suitable formulations for use in the present disclosure can be found, for example in Remington's Pharmaceutical Sciences 22nd edition, Allen, Loyd V., Jr editor (September 2012); and Akers, Michael J. Sterile Drug Products: Formulation, Packaging, Manufacturing and Quality; publisher Informa Healthcare (2010). which is incorporated by reference herein.

In some aspects, the at least one OCS is delivered in the form of a cream, gel, lotion, liquid, ointment, collodion, foam, paste, aerosol, spray solution, dispersion, solid stick, emulsion, microemulsion, eye drop, nose drop, ear drop, and the like, that can be formulated using suitable excipients, such as, for example, emulsifiers, surfactants, thickening agents, sunscreen agents, moisturizers, cooling agents, skin lightening agent, skin conditioning agents, skin protectants, emollients, humectants, colorants, and combinations of two or more thereof.

Suitable skin penetration enhancers can be, for example, sulfoxides, alcohols, fatty acids, fatty acid esters, polyols, amides, surfactants, terpenes, alkanones, and organic acids, among others. Specific examples of suitable sulfoxides include dimethylsulfoxide (DMSO) and decylmethylsulfoxide, among others. Suitable alcohols include alkanols such as ethanol, propanol, butanol, pentanol, hexanol, octanol, n-octanol, nonanol, decanol, 2-butanol, 2-pentanol, and benzyl alcohol; fatty alcohols, such as caprylic alcohol, decyl alcohol, lauryl alcohol, 2-lauryl alcohol, myristyl alcohol, cetyl alcohol, stearyl alcohol, oleyl alcohol, linoleyl alcohol, and linolenyl alcohol; isopropyl alcohol, and 2-(2-ethoxy)ethanol. Examples of suitable fatty acids include linear fatty acids such as valeric acid, heptanoic acid, pelagonic acid, caproic acid, capric acid, lauric acid, myristic acid, stearic acid, oleic acid, and caprylic acid; and branched fatty acids, such as isovaleric acid, neopentanoic acid, neoheptanoic acid, neononanoic acid, trimethyl hexanoic acid, neodecanoic acid, and isostearic acid. Examples of suitable fatty acid esters include aliphatic fatty acid esters such as isopropyl n-butyrate, isopropyl n-hexanoate, isopropyl n-decanoate, isopropyl myristate, isopropyl palmitate, and octyldodecyl myristate; alkyl fatty acid esters such as ethyl acetate, butyl acetate, methyl acetate, methylvalerate, methylpropionate, diethyl sebacate, and ethyl oleate; and diisopropyl adipate and dimethyl isosorbide. Examples of suitable polyols include propylene glycol, propylene glycol monolaurate, butylene glycol, polyethylene glycol, ethylene glycol, diethylene glycol, triethylene glycol, dipropylene glycol, ethoxydiglycol, pentylene glycol, glycerol, propanediol, butanediol, pentanediol, hexanetriol, and glycerin. Examples of suitable amides include urea, dimethylacetamide, diethyltoluamide, dimethylformamide (DMF), dimethyloctamide, dimethyldecamide, biodegradable cyclic urea (e.g., 1-alkyl-4-imidazoline-2-one), pyrrolidone derivatives, biodegradable pyrrolidone derivatives (e.g., fatty acid esters of N-(2-hydroxyethyl)-2-pyrrolidone), cyclic amides, hexamethylenelauramide and its derivatives, diethanolamine, and triethanolamine. Examples of pyrrolidone derivatives include 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-cyclohexylpyrrolidone, N-dimethylaminopropylpyrrolidone, N-cocoalkypyrrolidone, N-tallowalkylpyrrolidone, and N-methylpyrrolidone. Examples of cyclic amides include 1-dodecylazacycloheptane-2-one (e.g., Azone™), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyldodecyl)azacyclohaptane-2-one, 1-geranylazacyclohexane-2-one, 1-geranylazacyclopentan-2,5-dione, and 1-farnesylazacyclopentan-2-one. Other examples include lauryl lactate, caprylocaproyl polyoxyl-8 glyceride, polyglyceryl oleate, polyoxyethylated glycolysed glyceride, and lecithin isopropyl palmitate. In some aspects, the skin penetration enhancer is one or more of Laurogicol™ 90, ethanol, Transcutol® (diethylene glycol monoethyl ether), Labrasol® (PEG-8 caprylic/capric glycerides), Plurol® Oleique (Polyglyceryl-3 oleate), Labrafil® 2125cs, oleic acid, HPbCD, propylene glycol (PG), and lecithin isopropyl palmitate (LIPS). In some cases, the skin penetration enhancer also functions as a solvent.

In some cases, the skin penetration enhancer is present in the formulation in an amount ranging from about 1 wt % to about 98 wt %, such as 1 wt % to 90 wt %, 2 wt % to 50 wt %, 5 wt % to 50 wt %, or 7 wt % to 20 wt %, based on weight of the composition.

Exemplary thickening agents include but are not limited to: cetearyl alcohol, polyethylene glycol, polyethylene oxide, synthetic polymers and vegetable gums; cellulose derivatives (methylcellulose (MC), carboxymethylcellulose (CMC), hydroxypropylcellulose, hydroxypropyl methylcellulose), carbomers (polyacrylic acids such as Carbopol® 910, Carbopol® 941), cetearyl alcohol, magnesium aluminum silicate, acryloyldimethyl taurate copolymer, various multipblock copolymers, poloxamers (Pluronic®), various carboxylic acid polymers (e.g. acrylates), sulfonated polymers (e.g. sodium polyacryloyldimethyl taurate), clays, silicon dioxide, and copolymers, hydrophobically modified derivatives, and mixtures thereof. Gums, including natural gums, include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, fumed silica, hydroxypropyl chitosan, hydroxypropyl guar, karaya gum, kelp, locust bean gum, natto gum, potassium alginate, sodium alginate, potassium carrageenan, propylene glycol alginate, sclerotium gum, sodium carboxymethyl dextran, sodium carrageenan, tragacanth gum, xanthan gum, derivatives thereof and mixtures thereof. In some aspects, the thickening agent is one or more of polyacrylic acid, polyacrylic acid crosslinked with allyl sucrose (a Carbopol®), polyacrylic acid crosslinked with allyl pentaerythritol (a Carbopol®), polyacrylic acid and C10-C30 alkyl acrylate crosslinked with allyl pentaerythritol (a Carbopol®), poly(ethylene glycol)-block-poly(propylene glycol)-block-poly(ethylene glycol) (Lutrol® F127) or poloxamer 188 (Pluronic® F68).

Exemplary humectants include but are not limited to polyols. For instance, the humectant may comprise at least one of glycerin, propylene glycol, PEG, sorbitol solution, and 1,2,6 hexanetriol.

Exemplary pH adjusters include but are not limited to: adipic acid, aliphatic amine neutralizing agents (ethanolamine, triethanolamine, diisopropanolamine), alpha-ketoglutaric acid, 2-amino-2-methyl-1,3-propanediol, 2-amino-2-methyl-1-propanol, 1-amino-2-propanol, ammonium bicarbonate, ammonium phosphate, ascorbic acid, benzoic acid, calcium citrate, calcium hydroxide, citric acid, phosphoric acid, tartaric acid, sodium hydroxide, a phosphate, monobasic sodium phosphate, a carbonate, an acetate, sodium hydroxide, potassium hydroxide, trolamine, and the like. In some aspects, trolamine is used to adjust the pH. In some cases, the pH adjuster is a buffer.

Emollients are supple, waxlike, lubricating, thickening agent that prevents water loss and have a softening and soothing effect on skin. Examples of emollients are ingredients like plant oils, mineral oil, shea butter, cocoa butter, petrolatum, and fatty acids (animal oils, including emu, mink, and lanolin, the latter probably the one ingredient that is most like our own skin's oil). More technical-sounding emollient ingredients, such as triglycerides, benzoates, myristates, palmitates, and stearates, are generally waxy in texture and appearance but provide most moisturizers with their elegant texture and feel.

Exemplary emollients, e.g., for use in aqueous lotion compositions having a low pH and increased spreading and slip characteristics, include, but are not limited to, oleic acid, soy lecithin, C12-C15 alkyl benzoate, stearic acid, white wax, yellow wax, carnauba wax, cetyl ester wax, microcrystalline wax, paraffin wax, beeswax, caprylic/capric triglyceride, glycerin, glyceryl stearate, PEG-10 sunflower oil glycerides; vegetable oils like sunflower oil, palm oil, olive oil, emu oil, babassu oil, evening primrose oil, palm kernel oil, cottonseed oil, jojoba oil, meadowfoam seed oil, sweet almond oil, canola oil, soybean oil, avocado oil, safflower oil, coconut oil, sesame oil, rice bran oil, and grape seed oil; mineral oil; esters like isopropyl stearate, isostearyl isononanoate, diethylhexyl fumarate, diisostearyl malate, triisocetyl citrate, stearyl stearate, diglycol stearate, methyl palmitate, and methylheptyl isostearate; petrolatum; hydrous lanolin, lanolin oil, lanolin alcohol, and lanolin wax; long chain alcohols like cetyl alcohol, stearyl alcohol, behenyl alcohol, isostearyl alcohol, 2-hexyldecanol and myristyl alcohol; dimethicone fluids of various molecular weights and mixtures thereof; PPG-15 stearyl ether (also known as arlatone E); shea butter; olive butter; sunflower butter; coconut butter; jojoba butter; cocoa butter; squalane and squalene; isoparaffins; polyethylene glycols of various molecular weights; polypropylene glycols of various molecular weights; and mixtures thereof. In some aspects, Tween® and/or Span® is/are used as emollient(s).

Exemplary emulsifiers include, but are not limited to, poloxamer, emulsifying wax, sodium lauryl sulfate, propylene glycol monostearate, diethyl glycol monoethyl ether, docusate sodium, ethoxylated alcohols like laureth-23, ceteth-2, ceteth-10, ceteth-20, ceteth-21, ceteareth-20, steareth-2, steareth-10, steareth-20, steareth-21, oleth-2, oleth-10, oleth-20, steareth-100, steareth-21; ethoxylated alkylates like PEG stearate, PEG-8 stearate, PEG-40 stearate (i.e., polyoxy ethylene 40 stearate), PEG-2 stearate, PEG-50 stearate, PEG-20 palmitate, PEG-2 palmitate, and PEG-100 stearate; sorbitan monoalkylates like sorbitan stearate; sorbitan laurate; sorbitan oleate, and sorbitan palmitate; other alkylated sorbitans like sorbitan tristearate, sorbitan sesquioleate, and sorbitan trioleate; ethoxylated sorbitans like polysorbate 20, polysorbate 21, polysorbate 40, polysorbate 60, polysorbate 61, polysorbate 65, polysorbate 80, polysorbate 81, PEG-40 sorbitan peroleate, and polysorbate 85; PEG-40 hydrogenated castor oil (also known as Emulsogen HCW-049); citric acid esters (such as Citrem N12 Veg K from Danisco Inc.); lactic acid esters; acetic acid esters; alkyl polyglycosides; sulfosuccinates and sulfosuccinate derivatives such as sodium dioctyl sulfosuccinate; and mixtures thereof.

Exemplary preservatives include but are not limited to: imidurea, acids such as benzoic acid, sorbic acids, boric acids, etc; esters such as methylparaben, ethylparaben, propylparaben, butylparaben, sodium benzoate, sodium propionate, potassium sorbate, etc.; alcohols such as chlorobutanol, benzyl alcohol, phenyl ethyl alcohol, etc.; phenols such as phenol, chlorocresol, o-phenyl phenol, phenoxyethanol, etc.; mercurial compounds such as thiomersal, nitromersol, phenylmercuric nitrate, phenylmercuric acetate, etc.; and quaternary ammonium compounds such as benzalkonium chloride, cetyl pyridinium chloride, etc. and combination of these, e.g., a combination of methylparaben and propylparaben.

In some cases, the formulations of the present disclosure include a chelating agent, such as ethylene diamine tetraacetate.

In some cases, the formulations of the present disclosure include an antioxidant, such as butylated hydroxyanisole or butylated hydroxytoluene.

In some cases, the formulations of the present disclosure include a solvent, such as water, purified water, hexylene glycol, propylene glycol, oleyl alcohol, propylene carbonate, dimethylsulfoxide, N-methyl-pyrrolidone, and mineral oil. In some cases, the formulation includes a solvent in which the OCS is soluble. In some cases, the solvent also functions as a skin penetration enhancer. In other cases, the solvent does not function as a skin penetration enhancer. The solvent may be present in an amount ranging from about 1 wt % to about 98 wt %, such as about 2 wt % to about 75 wt %, 3 wt % to about 50 wt %, 4 wt % to about 25 wt %, and 5 wt % to about 10 wt %, based on weight of the formulation.

Those of skill in the art will recognize that some excipients may have more than one role or function in a composition. For example, polyethylene glycol may function as both a thickener and as an emollient.

In other aspects, the at least one OCS is transdermally administered in the form of a transdermal patch or iontophoresis device. Other components can optionally be incorporated into the transdermal patches. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Woven pads or rolls of bandaging material, e.g., gauze, can be impregnated with the compositions in solution, lotion, cream, ointment or other such form can also be used for topical application. In one embodiment, the compositions of the present disclosure are administered as a transdermal patch. In another embodiment compositions of the present disclosure are administered as a sustained-release transdermal patch. The transdermal patches of the present disclosure can include, for example, adhesive matrix, polymeric matrix, reservoir patch, matrix or monolithic-type laminated structure, and are generally comprised of one or more backing layers, adhesives, penetration enhancers, an optional rate controlling membrane and a release liner which is removed to expose the adhesives prior to application. Polymeric matrix patches also comprise a polymeric-matrix forming material.

In one aspect, the OCS is combined with a standard USP hydrophilic ointment; a thousand grams of which contains the following compounds in the indicated amounts:

Methylparaben 0.25 g

Propylparaben 0.15 g

Sodium lauryl sulfate 10 g

Propylene glycol 120 g

Stearyl alcohol 250 g

White petrolatum 250 g

Purified water 370 g

The ingredients of hydrophilic ointment USP, which ointment is commonly available from a variety of commercial sources, may be combined as follows. First, the stearyl alcohol and the white petrolatum are melted on a steam bath and warmed to about 75° C. The other ingredients are dissolved in the purified water and are also warmed to about 75° C. All ingredients are then mixed together and stirred until the mixture congeals.

It will be understood that the hydrophilic ointment disclosed above is given by way of example only, and that numerous other carriers may also be suitable, such as an oleic acid ointment base.

In another exemplary aspect, the composition comprises one or more of water, mineral oil (paraffinum liquidum), glyceryl stearate SE, propylene glycol, stearic acid, isopropyl myristate, isopropyl palmitate, cetyl esters, propylene glycol stearate SE, tocopheryl acetate (vitamin E acetate e.g. about 12,000 I.U. of vitamin E), cetyl alcohol, mineral oil and lanolin alcohol (e.g., paraffinum liquidum and lanolin alcohol), stearyl alcohol, triethanolamine, titanium dioxide, trisodium EDTA, diazolidinyl urea, methylparaben, propylparaben, and sodium benzoate.

In some aspects, the pharmaceutical formulation is (a) a lotion or cream, or (b) a controlled release formulation, or (c) a suspension. A suspension is a preferred aspect of the present disclosure.

Controlled release refers to the presentation or delivery of compounds in response to time, and commonly refers to time dependent release. Controlled release has several variants such as sustained release (where prolonged release is intended), pulsed release (bursts of drug are released at different times), delayed release (e.g. to target different regions of the gastrointestinal tract), etc. Controlled release formulations may prolong drug action and maintain drug levels within a desired therapeutic window to avoid potentially hazardous peaks in drug concentration following ingestion or injection, and to maximize therapeutic efficiency. In addition to pills, capsules and injectable drug carriers (that may have an additional release function), forms of controlled release medicines include gels, implants, devices and transdermal patches.

In some aspects, the formulations of the present disclosure are made by combining the at least one OCS with vehicle. In other aspects, the formulations are made by dissolving drug in a penetration enhancer and then adding other excipients, such as one or more thickening agents. In a composition that comprises a skin penetration enhancer and a thickening agent, the thickening agent is typically different from the skin penetration enhancer.

Each excipient of the at least one pharmaceutically acceptable excipient, when present, is typically present in a percentage of from e.g. about 1 to about 99%, for example, about 10 to about 90%, e.g. about 10, 15, 20, 25, 30, 35, 40, 45, 50, 55, 60, 65, 70, 75, 80, 85, 90, or 95%, in terms of weight percentage of a total formulation, or in terms of volume percentage of the total formulation, as appropriate.

The final amount of OCS in a formulation may also vary but in general will be from about 1-99% (w/w). Depending on the formulation, it is expected that the active components (e.g. at least one OCS) will be present as about 0.1% to about 99% (w/w) of the composition, or about 0.5 to 50%, 0.5 to 20%, 1 to 80%, or about 10 to 50% (w/w), and the vehicular “carrier” will constitute about 1% to about 99.9% (w/w) of the composition. The pharmaceutical compositions of the present disclosure may include any suitable pharmaceutically acceptable additives or adjuncts to the extent that they do not hinder or interfere with the therapeutic effect of the OCS(s).

In some aspects, if a single (only one) OCS (e.g. 25HC3S or 25HCDS) is present in a liquid, lotion, or cream composition (including liquid solutions, suspensions such as liquid suspensions, lotions, creams, etc.), the concentration of the OCS generally ranges from about 0.01 to about 200 mg/ml, or from about 0.1 to 100 mg/ml, and is generally from about 1 to about 50 mg/ml, e.g. is about 1, 5, 10, 15, 20, 25, 30, 35, 40, 45, or 50 mg/ml. If multiple OCS's are present (e.g. 2 or more, such as 2, 3, 4, 5, or more) are present in a liquid, lotion, or cream composition, the concentration of each typically ranges from about 0.01 to about 200 mg/ml, or from about 0.1 to 100 mg/ml, and generally from about 1 to about 50 mg/ml, e.g. is about 1, 5, 10, 15, 20, 25, 30, 35, 40, 45, or 50 mg/ml.

In some aspects, if a single (only one) OCS (e.g. 25HC3S or 25HCDS) is present in a solid or semi-solid composition (e.g. a gel or other solidified preparation), the concentration of the OCS generally ranges from about 0.01 to about 75% (w/w) or from about 0.1 to about 50% (w/w), and is generally from about 1 to about 25% (w/w), e.g. is about 1, 5, 10, 15, 20, 25, 30, 35, 40, 45, or 50% (w/w). If multiple OCS's are present (e.g. 2 or more, such as 2, 3, 4, 5, or more) in a solid or semi-solid composition, the concentration of each typically ranges from about 0.01 to about 75% (w/w) or from about 0.1 to about 50% (w/w), and is generally from about 1 to about 25% (w/w), e.g. is about 1, 5, 10, 15, 20, 25, 30, 35, 40, 45, or 50% (w/w).

In some aspects, if a single (only one) OCS (e.g. 25HC3S or 25HCDS) is present in a lyophilized solid composition (e.g. for reconstitution with a carrier before administration), the concentration of the OCS generally ranges from about 0.01 to about 75% (w/w), about 0.1 to about 50% (w/w), and is generally from about 1 to about 15% (w/w), e.g. is about 1, 2, 3, 4, 5, 6, 7, 8, 9, 10, 11, 12, 13, 14, or 15% (w/w). If multiple OCS's are present (e.g. 2 or more, such as 2, 3, 4, 5, or more) in a lyophilized solid composition, the concentration of each typically ranges from about 0.01 to about 75% (w/w), about 0.1 to about 50% (w/w), and is generally from about 1 to about 15% (w/w), e.g. is about 1, 2, 3, 4, 5, 6, 7, 8, 9, 10, 11, 12, 13, 14, or 15% (w/w).

In some aspects, the formulations comprise one or more OCSs as described herein, together with propylene glycol and/or cyclodextrin. The propylene glycol, when present, is present in a v/v percentage of from e.g. about 1 to about 99%, for example, about 10 to about 90%, e.g. about 10, 15, 20, 25, 30, 35, 40, 45, 50, 55, 60, 65, 70, 75, 80, 85, 90, or 95%, in terms of volume percentage of a total formulation.

In some aspects, CD is present in a liquid and/or solution product in a range of from about 1 to about 65% (w/v), e.g. about 1, 2, 3, 4, 5, 10, 20, 30 or 40% (w/v). In some aspects, the amount is 25% (w/v). In some aspects, CD is present in a lyophilized solid product (e.g. for reconstitution) in a range from about 1 to about 90% (w/w), e.g. about 1, 5, 10, 40, 50, 60, 70, 80 or 90% (w/w). In some aspects, the amount is 89% (w/w). In some aspects, CD is present in a solid product for administration in a range from about 1 to about 90% (w/w), e.g. about 1, 5, 10, 40, 50, 60, 70, 80 or 90% (w/w). In some aspects, the amount is 89% (w/w).

In many cases, high water content reduces solubility of the OCS, e.g., 25HC3S. In some cases, in order to increase concentration of 25HC3S water is excluded or limited in the compositions, and silicon dioxide is used as a thickener to form a gel. In some aspects, water is present in the composition in an amount ranging from about 0.5 wt % to about 90 wt %, such as about 50 wt % to 90 wt %, about 1 wt % to about 10 wt %, or about 1 wt % to about 5 wt %, based on weight of the composition.

In some aspects, the composition is contained within a vial, e.g., a glass vial. In other aspects, the composition is contained within a tube or pump dispenser. In still other aspects, the composition is contained within an aerosol or spray container.

### Administration

Implementation of the methods generally involves identifying patients suffering from or at risk of developing inflammatory skin disease or skin lesion, or a condition associated with inflammatory skin disease or skin lesion, and administering one or more OCS in an acceptable form by an appropriate route. Prophylactic treatments are also encompassed and include, for example, administration after a known or suspected exposure to an etiological agent (e.g. poison ivy), and/or at very early stages of disease; or in a subject who has had symptoms of a disease that have dissipated but for which a reoccurrence is possible, or who has known risk factors (such as a genetic predisposition, past exposure to a noxious agent that causes skin inflammation, skin lesions, etc.), and the like.

The compositions (preparations) of the present disclosure are formulated for and administered by any of the many suitable means which are known to those of skill in the art, including but not limited to: topically, orally or parenterally, including intravenously, intramuscularly, subcutaneously, by intradermal injection, by subdermal injection, by intralesional injection, by intraperitoneal injection, etc., or by other routes such as transdermal, sublingual, rectal and buccal delivery, inhalation of an aerosol, intravaginally, intranasally, via various drops (such as eye drops) and sprays, preparations for insufflation, or via direct subcutaneous delivery at the affected area, etc. In some aspects, the route of administration depends on the nature or stage of the condition that is treated, e.g. on the type or degree of inflammatory skin disease, etc. Administration may be local or systemic.

In some aspects, the pharmaceutical composition that is used in the methods of the present disclosure is formulated for topical administration, including administration directly to the skin or a membrane of a subject, for example, at an area requiring treatment. Pharmaceutical compositions for topical administration may, for example, be in the form of solutions, creams, ointments, jellies, gels, sprays, foams, powders, liposomes, or aqueous or oily solutions or suspensions, liquids, etc., that is rubbed, sprayed or “painted” onto the skin or membrane. Further, the active agent(s) may be impregnated into a delivery device such as a bandage which covers the affected area.

In the case of topical application to the scalp, the pharmaceutical composition may be formulated as a shampoo. In the case of topical application to the skin, the pharmaceutical composition may be formulated as an additive to wash water (for example in the form of a bath or shower gel or cream), such as to bath water etc. Such pharmaceutical compositions for topical administration may include diluents or carriers that are also suitable for use in cosmetics. Pharmaceutical compositions for topical administration by application to the skin may include moisturizers, and sun tan, sun screen and sunblock lotions and creams.

A pharmaceutical composition for topical administration may be provided in a suitable container, such as a pipette, for direct administration in one or two spots to the skin, for example for administration to a pet such as a dog or cat. For example, a pipette may be provided with a snap-off top and containing a single dosage of the active ingredient, such that direct administration of the whole contents of the pipette in one or two spots to the skin provides a desired dosage of the active ingredient.

Alternatively, the topical administration may be achieved by means of diffusion from or through a suitable material to the skin, i.e. wherein the active ingredient is releasably contained in or applied to the material for release to the skin upon contact therewith. For example, suitable materials may be provided in the form of a bandage, as gloves, socks, etc.

In some aspects, oral administration is particularly effective when used prophylactically, e.g. to prevent inflammatory skin disease or skin lesions. In some aspects, when damage has already occurred, and especially when inflammatory skin disease and/or a skin lesion is diagnosed, the route of administration is generally topical, subcutaneous or intradermal.

The subject to whom the OCS is administered is generally a mammal, frequently a human, but this is not always the case. Veterinary applications of this technology are also contemplated, e.g. for companion pets (cats, dogs, etc.), or for livestock and farm animals, for horses, and even for “wild” animals that have special value or that are under the case of a veterinarian, e.g. animals in preserves or zoos, injured animals that are being rehabilitated, etc.

In some aspects, the compositions are administered in conjunction with other therapies and treatment modalities such as various pain relief medications, anti-arthritis agents, various chemotherapeutic agents, allergy treatments (e.g. anti-histamines), phototherapy, antibiotic agents, diet regimens (e.g. diet restrictions), steroids, and the like, depending on the malady with which the subject is afflicted. “In conjunction with” refers to both administration of a separate preparation of, or treatment with the one or more additional agents during or overlapping with, the course of treatment with the compositions described herein, and also to inclusion of the one or more additional agents in a composition of the present disclosure.

In some cases, the OCS composition is administered prophylactically or therapeutically to an individual prior to, simultaneously (concurrently) with or sequentially with other therapeutic regimens or agents (e.g., multiple drug regimens, adjuvant therapy), including with other therapeutic regimens or medications that are use in treating, for example, psoriasis and/or skin lesions. Medications (i.e., drugs) suitable for combination therapies in accordance with the present disclosure include pain medications (analgesics), including but not limited to acetaminophen, codeine, propoxyphene napsylate, oxycodone hydrochloride, hydrocodone bitartrate and tramadol; biologics such as adalimumab and etanercept: methotrexate; leflunomide (original brand name Arava®); sulfasalazine; cyclosporine; gold salts; azathioprine; antimalarials; oral steroids (e.g. prednisone); colchicine; non-steroidal anti-inflammatories, including but not limited to salicyclic acid (aspirin), ibuprofen, indomethacin, celecoxib, rofecoxib, ketorolac, nambumetone, piroxicam, naproxen, oxaprozin, sulindac, ketoprofen, diclofenac, other COX-1 and COX-2 inhibitors, salicyclic acid derivatives, propionic acid derivatives, acetic acid derivatives, fumaric acid derivatives, carboxylic acid derivatives, butyric acid derivatives, oxicams, pyrazoles and pyrazolones. Other agents suitable for use in combination with the one or more OCS include topical steroids, systemic steroids, glucocorticoids, antagonists of inflammatory cytokines, antibodies against T cell surface proteins, anthralin, coal tar, vitamin D analogs (including vitamin D3 and its analogs e.g. 1,25-dihydroxy vitamin D3 and calcipotriene), topical retinoids, oral retinoids (including but not limited to etretinate, acitretin and isotretinoin), topical salicylic acid, hydroxyurea, minocycline, misoprostol, oral collagen, penicillamine, 6-mercaptopurine, nitrogen mustard, gabapentin, bromocriptine, somatostatin, peptide T, anti-CD4 monoclonal antibody, fumaric acid, polyunsaturated ethyl ester lipids, zinc, topical oils (including fish oils, nut oils and vegetable oils), aloe vera, topical jojoba, topical Dead Sea salts, topical capsaicin, topical milk thistle, topical witch hazel, moisturizers and topical Epson salts. Therapeutic regimens suitable for use in combination with the one or more OCS for treating psoriasis and/or skin lesions include but are not limited to plasmapheresis, phototherapy with ultraviolet light B, psoralen combined with ultraviolet light A (PUNA), photochemotherapy and sunbathing. When the one or more OCS is administered simultaneously with other therapeutic agents, they can be administered in the same or different compositions.

The administration of the compositions of the present disclosure is at any suitable frequency commensurate with the type of formulation and the condition being treated. For example, if a topical formulation is utilized, administration generally ranges from about 1 to about 5 times a day, or once every few days, or once per week, or once per month, etc. Administration may also be on an “as needed” basis. In addition, in some aspects, a combination of administration modes is utilized, e.g. intradermal or subcutaneous injections may initially be used, followed by less-invasive, self-administered topical treatment as symptoms subside, followed by injections in the case of a “flare” of symptoms, etc. Alternatively, topical treatment may be used exclusively. In addition, the time of day and the number of times per day that the pharmaceutical formulation is administered may vary and are best determined by a skilled practitioner such as a physician. In some aspects, formulations are administered from three times daily to annually, such as twice daily to annually, daily to annually, daily to half yearly, daily to quarterly, daily to monthly, or daily to weekly. As discussed in Example 5, for several patients the areas treated with a single injection of 25HC3S in suspension were observed 4 to 9 months after the injection. In at least some of these patients, the treated area appeared to have less psoriasis. In at least some of these patients, the untreated area also appeared to have less psoriasis.

In some cases, the dose administered is in the range of from about 1 mg/cm² to about 5000 mg/cm², e.g. about 10 mg/cm² to about 1000 mg/cm². A desirable local exposure of OCS in or at a surface area of skin or membrane that is being treated may be in the range of from about 0.01 mg/cm² to about 50 mg/cm², e.g. about 0.1 to about 10 mg/cm². Topical or intralesional doses generally range from about 1 milligram to about 50,000 milligrams of OCS, such as 25HC3S or a pharmaceutically acceptable salt thereof, per person per day. In some aspects, the dose is from about 10 milligrams to about 2000 milligrams per person per day, or about 100 milligrams to about 1000 milligrams per person per day. Oral and injectable delivery forms generally utilize, e.g. dosages in the range of from about 0.001 to about 100 mg or more of compound per kg of body weight per 24 hr., and preferably about 0.01 to about 50 mg of compound per kg of body weight per 24 hr., and more preferably about 0.1 to about 10 mg of compound per kg of body weight per 24 hr. Daily non-topical doses generally range from about 0.1 milligrams to about 5000 milligrams of OCS, such as 25HC3S or a pharmaceutically acceptable salt thereof, per person per day. In some aspects, the dose is from about 10 milligrams to about 2000 milligrams per person per day, or about 100 milligrams to about 1000 milligrams per person per day. In some aspects, the exact dosage to be administered varies depending on the nature of the malady that is being prevented or treated, the route of administration, the bioavailability, the particular formulation that is administered, the age, gender, weight and overall health status of the individual patient, the precise etiology of the disease, the extent or progression of the disease or condition being treated, and on whether the treatment is prophylactic or intended to effect a cure.

The OCS is generally administered in forms not naturally found in the body, and in concentrations that are significantly higher than those which occur naturally. For example, for 25HC3S, natural levels typically range from e.g. about 2 ng/ml or less up to about 5 ng/ml in plasma. The concentration of OCS (e.g. 25HC3S) in the blood or plasma of a patient that is treated with an OCS (e.g. 25HC3S) is generally greater than about 5 ng/ml, and generally ranges from about 50 ng/ml to about 5000 ng/ml, such as about 80 ng/ml to about 3000 ng/ml, e.g. from about 100 to about 2000 ng/ml, or from about 200 to about 1000 ng/ml.

### Secondary Conditions and Patient Populations

In addition to exhibiting skin inflammation, in some aspects, the populations of subjects treated by the methods described herein may or may not have symptoms of and/or been diagnosed with high levels of cholesterol (hypercholesterolemia, e.g. cholesterol levels in serum in the range of about 200 mg/dl or more), or with a condition associated with high levels of cholesterol e.g. hyperlipidemia, atherosclerosis, heart disease, stroke, Alzheimer's, gallstone diseases, cholestatic liver diseases, etc. In some aspects, the populations of subjects treated by the methods described herein do not have symptoms of and/or have not been diagnosed with high levels of cholesterol (hypercholesterolemia, e.g. cholesterol levels in serum in the range of about 200 mg/dl or more), or with a condition associated with high levels of cholesterol e.g. hyperlipidemia, atherosclerosis, heart disease, stroke, Alzheimer's, gallstone diseases, cholestatic liver diseases, etc.

In further aspects, the populations of subjects treated by the methods described herein may or may not have symptoms of and/or been diagnosed with liver disorders such as hepatitis, inflammation of the liver, caused mainly by various viruses but also by some poisons (e.g. alcohol); autoimmunity (autoimmune hepatitis) or hereditary conditions; non-alcoholic fatty liver disease, a spectrum in disease, associated with obesity and characterized by an abundance of fat in the liver, which may lead to hepatitis, i.e. steatohepatitis and/or cirrhosis; cirrhosis, i.e. the formation of fibrous scar tissue in the liver due to replacing dead liver cells (the death of liver cells can be caused, e.g. by viral hepatitis, alcoholism or contact with other liver-toxic chemicals); haemochromatosis, a hereditary disease causing the accumulation of iron in the body, eventually leading to liver damage; cancer of the liver (e.g. primary hepatocellular carcinoma or cholangiocarcinoma and metastatic cancers, usually from other parts of the gastrointestinal tract); Wilson's disease, a hereditary disease which causes the body to retain copper; primary sclerosing cholangitis, an inflammatory disease of the bile duct, likely autoimmune in nature; primary biliary cirrhosis, an autoimmune disease of small bile ducts; Budd-Chiari syndrome (obstruction of the hepatic vein); Gilbert's syndrome, a genetic disorder of bilirubin metabolism, found in about 5% of the population; glycogen storage disease type II; as well as various pediatric liver diseases, e.g. including biliary atresia, alpha-antitrypsin deficiency, alagille syndrome, and progressive familial intrahepatic cholestasis, etc. In addition, liver damage from trauma may or may not be treated, e.g. damage caused by accidents, gunshot wounds, etc. Further, liver damage caused by certain medications may or may not be prevented or treated, for example, drugs such as the antiarrhythmic agent amiodarone, various antiviral drugs (e.g. nucleoside analogues), aspirin (rarely as part of Reye's syndrome in children), corticosteroids, methotrexate, tamoxifen, tetracycline, etc. are known to cause liver damage. In further aspects, the populations of subjects treated by the methods described herein do not have symptoms of and/or have not been diagnosed with liver disorders such as hepatitis, inflammation of the liver, caused mainly by various viruses but also by some poisons (e.g. alcohol); autoimmunity (autoimmune hepatitis) or hereditary conditions; non-alcoholic fatty liver disease, a spectrum in disease, associated with obesity and characterized by an abundance of fat in the liver, which may lead to hepatitis, i.e. steatohepatitis and/or cirrhosis; cirrhosis, i.e. the formation of fibrous scar tissue in the liver due to replacing dead liver cells (the death of liver cells can be caused, e.g. by viral hepatitis, alcoholism or contact with other liver-toxic chemicals); haemochromatosis, a hereditary disease causing the accumulation of iron in the body, eventually leading to liver damage; cancer of the liver (e.g. primary hepatocellular carcinoma or cholangiocarcinoma and metastatic cancers, usually from other parts of the gastrointestinal tract); Wilson's disease, a hereditary disease which causes the body to retain copper; primary sclerosing cholangitis, an inflammatory disease of the bile duct, likely autoimmune in nature; primary biliary cirrhosis, an autoimmune disease of small bile ducts; Budd-Chiari syndrome (obstruction of the hepatic vein); Gilbert's syndrome, a genetic disorder of bilirubin metabolism, found in about 5% of the population; glycogen storage disease type II; as well as various pediatric liver diseases, e.g. including biliary atresia, alpha-1 antitrypsin deficiency, alagille syndrome, and progressive familial intrahepatic cholestasis, etc. In addition, in some cases, the patients treated by the methods herein do not have liver damage from trauma, e.g. damage caused by accidents, gunshot wounds, etc. Further, in some cases, the patients treated by the methods herein do not have liver damage caused by certain medications, for example, drugs such as the antiarrhythmic agent amiodarone, various antiviral drugs (e.g. nucleoside analogues), aspirin (rarely as part of Reye's syndrome in children), corticosteroids, methotrexate, tamoxifen, tetracycline, etc. are known to cause liver damage.

In further aspects, the populations of subjects treated by the methods described herein may or may not have symptoms of non-alcoholic fatty liver disease (NAFLD) and/or nonalcoholic steatohepatitis (NASH). In further aspects, the populations of subjects treated by the methods described herein do not have symptoms of non-alcoholic fatty liver disease (NAFLD) and/or nonalcoholic steatohepatitis (NASH).

In yet further aspects, the populations of subjects treated by the methods described herein may or may not have symptoms of inflammatory bowel diseases and/or diabetes (e.g. type 2 adult onset diabetes). In further aspects, the populations of subjects treated by the methods described herein do not have symptoms of inflammatory bowel diseases and/or diabetes (e.g. type 2 adult onset diabetes).

In yet further aspects, the populations of subjects treated by the methods described herein may or may not have symptoms of leptin deficiency and/or leptin resistance and/or a lipid storage disease. These subjects may or may not have i) a genetic mutation that causes low levels of leptin production, or production of a non- or poorly functioning leptin molecule, such as occurs in leptin deficiency (LD) (e.g. a mutation in the LEP gene encoding leptin); or ii) a defect in leptin signaling, caused by e.g. a congenital or acquired abnormality or deficiency in the functioning of the leptin receptor, e.g. due to a genetic mutation of the leptin receptor (e.g. mutations in the Ob (lep) gene that encodes the leptin receptor) or due to an acquired loss of receptor sensitivity to leptin binding such as that which occurs in leptin resistance (LR); or iii) a lipid storage disorder, which are generally congenital. Lipid storage disorders include, for example, neutral lipid storage disease, Gaucher disease, Niemann-Pick disease, Fabry disease, Farber's disease, gangliosidoses such as GM1 gangliosidoses and GM2 gangliosidoses (e.g. Tay-Sachs disease and Sandhoff disease), Krabbé disease, metachromatic leukodystrophy (MLD, including late infantile, juvenile, and adult MLD), and acid lipase deficiency disorders such as Wolman's disease and cholesteryl ester storage disease. In further aspects, the populations of subjects treated by the methods described herein do not have symptoms of leptin deficiency and/or leptin resistance and/or a lipid storage disease. These subjects may or may not have i) a genetic mutation that causes low levels of leptin production, or production of a non- or poorly functioning leptin molecule, such as occurs in leptin deficiency (LD) (e.g. a mutation in the LEP gene encoding leptin); or ii) a defect in leptin signaling, caused by e.g. a congenital or acquired abnormality or deficiency in the functioning of the leptin receptor, e.g. due to a genetic mutation of the leptin receptor, (e.g. mutations in the Ob (lep) gene that encodes the leptin receptor) or due to an acquired loss of receptor sensitivity to leptin binding such as that which occurs in leptin resistance (LR); or iii) a lipid storage disorder, which are generally congenital. Lipid storage disorders include, for example, neutral lipid storage disease, Gaucher disease, Niemann-Pick disease, Fabry disease, Farber's disease, gangliosidoses such as GM1 gangliosidoses and GM2 gangliosidoses (e.g. Tay-Sachs disease and Sandhoff disease), Krabbé disease, metachromatic leukodystrophy (MLD, including late infantile, juvenile, and adult MLD), and acid lipase deficiency disorders such as Wolman's disease and cholesteryl ester storage disease.

In yet further aspects, the populations of subjects treated by the methods described herein may or may not have symptoms of organ failure or dysfunction, for example, failure or dysfunction of the heart, lungs (e.g., lungs damaged by pulmonary fibrosis, e.g., associated with chronic asthma), liver, pancreas, kidneys, brain, intestines, colon, thyroid, etc., e.g., caused by sepsis and/or by ischemia, including acute organ failure. In yet further aspects, the populations of subjects treated by the methods described herein do not have symptoms of organ failure or dysfunction, for example, for example, failure or dysfunction of the heart, lungs (e.g., lungs damaged by pulmonary fibrosis, e.g., associated with chronic asthma), liver, pancreas, kidneys, brain, intestines, colon, thyroid, etc., e.g., caused by sepsis and/or by ischemia, including acute organ failure.

The present invention will be further illustrated by way of the following Examples. These Examples are non-limiting and do not restrict the scope of the invention. Unless stated otherwise, all percentages, parts, etc. presented in the Examples are by weight.

### Examples

### Example 1. Injection Studies

Injection studies were conducted as follows: I. an acute (single dose) intramuscular (IM) injection study in rats; II. a two-week subcutaneous (SC) injection study in rats; and III. a two-week SC injection study in dogs.

### I. Acute Single Dose Study

For the acute single dose study, Hannover Wistar rats (n=5/sex/dose group) received a single IM injection followed by 2 and 14 day observation periods. The solution that was tested included 30 mg/mL of 25HC3S sodium salt in vehicle (250 mg/mL hydroxypropyl-β-cyclodextrin in 10 mM sodium phosphate buffer in sterile water). Dose levels of 0 (vehicle), 3, 10 and 30 mg/kg of 25HC3S sodium salt were administered in dose volumes of 1.0, 0.1, 0.3 and 1.0 mL/kg. The results showed minimal to moderate muscle degeneration/regeneration, hemorrhage and inflammation in injected muscles of incidence and severity similar in vehicle and drug-treated rats. The changes were less severe (minimal only) after 14 days, indicating partial recovery; no clear effect of the presence of 25HC3S or vehicle volume was observed. It was concluded that 25HC3S solution was well tolerated and that the local changes were likely due to the effect of injection (needle) trauma and/or vehicle.

### II. Two-Week SC Injection Study in Rats

In a separate study, Hannover Wistar rats (n=12/sex/dose group) received daily SC injections of a solution of 30 mg/mL of 25HC3S sodium salt in vehicle (250 mg/mL hydroxypropyl-β-cyclodextrin in 10 mM sodium phosphate buffer in sterile water) for 2 weeks. Dose levels of 0 (vehicle), 15, 45 and 150 mg/kg of 25HC3S sodium salt were administered in dose volumes of 5.0, 0.5, 1.5 and 5.0 mL/kg. Following 14 days of dose administration, all rats were euthanized and necropsied

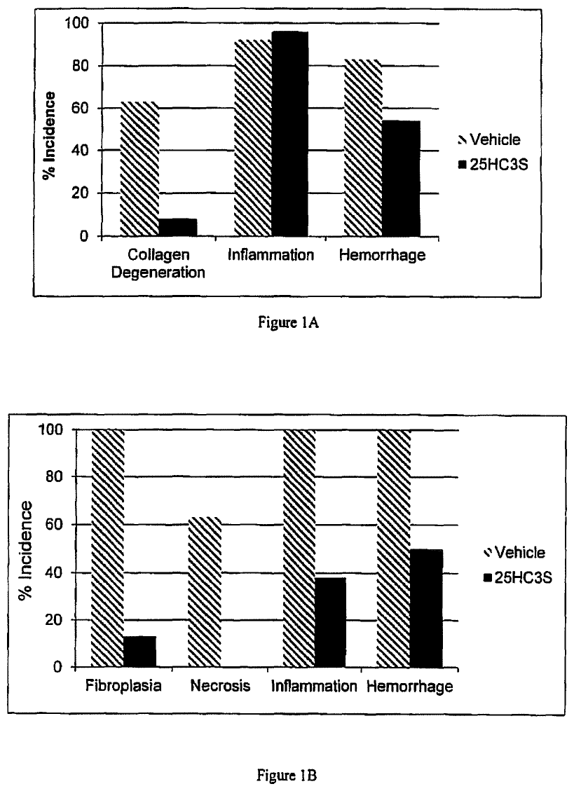

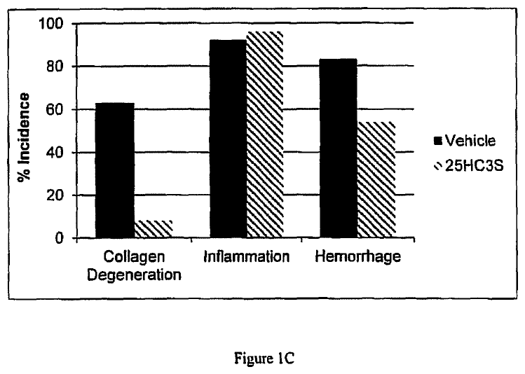

The results showed lower (22%) mean serum cholesterol in males given 150 mg/kg 25HC3S compared to vehicle controls after 2 weeks and higher (10%) mean liver weights in the 150 mg/kg 25HC3S males and females compared to controls. Cytoplasmic vacuolation of the proximal tubules of the kidneys was observed in vehicle controls and in the highest-dosed rats (150 mg/kg) as well; the severity was similar in vehicle control and rats. A minimal increase in alveolar macrophages in the lungs of vehicle controls and drug-treated rats was noted, as was collagen degeneration, hemorrhage, inflammation, and necrosis/degeneration of panniculus muscle at the injection sites of vehicle and drug-treated rats. However, as shown in FIG. 1A, collagen degeneration and hemorrhage tended to be lower in rats receiving 25HC3S compared to vehicle.

### III. Two-Week SC Injection Study in Dogs

In a separate study, Beagle dogs (n=4/sex/dose group) received daily SC injections for 2 weeks. The solution that was tested included 30 mg/mL of 25HC3S sodium salt in vehicle (250 mg/mL hydroxypropyl-β-cyclodextrin in 10 mM sodium phosphate buffer). Dose levels of 0 (vehicle), 3, 10 and 30 mg/kg of 25HC3S were administered in dose volumes of 1.0, 0.1, 0.33 and 1.0 mL/kg. Following 14 days of dose administration, all dogs were euthanized and necropsied. The results showed fibroplasia, hemorrhage, inflammation and necrosis in vehicle and drug-treated injection sites; the incidence and severity were generally higher in vehicle controls compared to drug-treated dogs (see FIG. 1B). In addition, swelling at the site of injection was markedly decreased in dogs receiving 25HC3S, compared to those receiving only vehicle (FIG. 1C).

The reduction in inflammation, necrosis, and hyperplasia suggests that 25HC3S may reduce inflammation, necrosis, and hyperplasia.

### Example 2. Evaluation of the Anti-Inflammatory Activity of 25HC3S Administered Intradermally in an Imiquimod (IMQ)-Induced Psoriasis Mouse Model

### Materials and Methods

### Animals

The subjects for the study were 40 male Balb/C mice (18-22 g). Animals exhibiting no signs of clinical distress, disease or injury during a 72-hr quarantine period were accepted for the study and received routine animal care throughout. The backs of all mice were shaved for an area of 1.5 cm×2 cm.

### Formulations

Two formulations of 25HC3S, Formulation A and Formulation B, were used for the study.

Formulation A was a clear solution of 25 HC3S sodium salt (30 mg/mL) in a solution vehicle (250 mg/mL hydroxypropyl betadex (beta cyclodextrin, 2-hydroxypropyl ether, a partially substituted poly(hydroxypropyl) ether of beta cyclodextrin) and 10 mM sodium phosphate buffer in sterile water). Vehicle was stored at 2-8° C. storage and placed at room temperature for 30 min. prior to mixing with powdered 25HC3S just prior to use. Dissolution of the 25HC3S in Vehicle A was rapid and appeared to be complete upon mixing.

Formulation B was a milky suspension of 25HC3S sodium salt (25 mg/mL) in a suspension vehicle (30 mg/mL polyethylene glycol 3350, 3 mg/mL polysorbate 80, 7.5 mg/mL NaCl, and 10 mM sodium phosphate buffer in sterile water). The 25HC3S was milled using a Fluid Energy Model 00 Jet-O-Mizer™ to approximately a 5 microns average particle size (measured by a Malvern Mastersizer 2000 equipped with a hydro 2000S dispersion cell) prior to addition to the vehicle. Vehicle was stored at 2-8° C. storage and placed at room temperature for 30 min. prior to mixing with powdered 25HC3S just prior to use. Because Formulation B is a suspension, the following mixing protocol was used: 3.0 mL of suspension vehicle was added to a vial containing pre-weighed powdered 25HC3S. The vial was shaken for 15 minutes on a flatbed shaker to create a uniformly white suspension, and then manually inverted 5-10 times, and shaken for 5 more minutes. In addition, immediately before administration, the vial was manually inverted 5-10 times to ensure uniformity of suspension.

### Administration of IMQ, Vehicle and 25HC3S

IMQ was applied topically once daily in the morning to the shaved back skin (50 mg) and right ear (25 mg) of each mouse in order to induce psoriasis-like conditions for 6 days (Day 0-5).

The 25HC3S in vehicle or the vehicle alone (N=10 mice/group) were administered once during the afternoons of Days 1 and 4 by intradermal injection. Injections were done approximately 6 hours after the day's IMQ application. Intradermal injections (50 μL/mouse) were given into the site of the back skin lesion.

For the solution formulation, treated mice were given a dose of 1.5 mg of 25HC3S each day, while in the suspension group, treated mice were given a dose of 1.25 mg of 25HC3D per injection.

### Monitoring and Measuring Parameters

Mice were monitored for signs of distress and daily photos of the back lesions were taken. Erythema, scaling, and thickness of the back skin was scored daily on a scale from 0 to 4 by an independent scorer (blind), where 0=none; 1=slight; 2=moderate; 3=marked; and 4=very marked. A cumulative score (erythema+scaling+thickening) was calculated as an indicator of the severity of the inflammation (on a scale of 0-12). Back skin thickness was measured by electronic calipers as an indicator of edema.

### Termination (Day 6)

All mice in the study were anesthetized and exsanguinated. The blood was collected, processed to sera and stored at −80° C. for analytical use.

### Cytokine Analysis

Half of the back skin was homogenized for measurement of cytokines TNFα and IL-17 by ELISA.

### Results

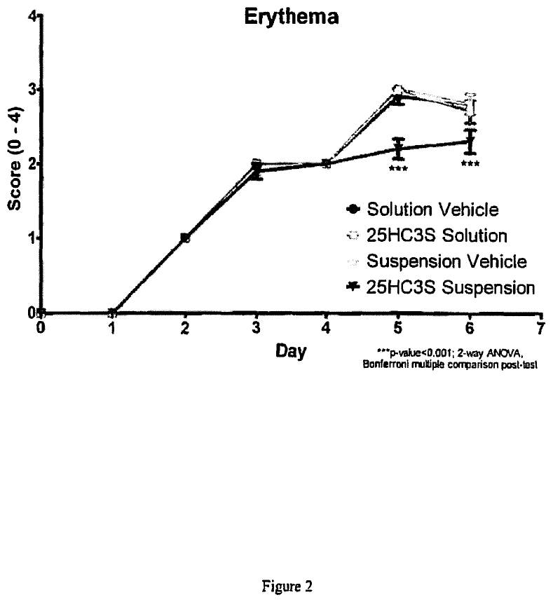

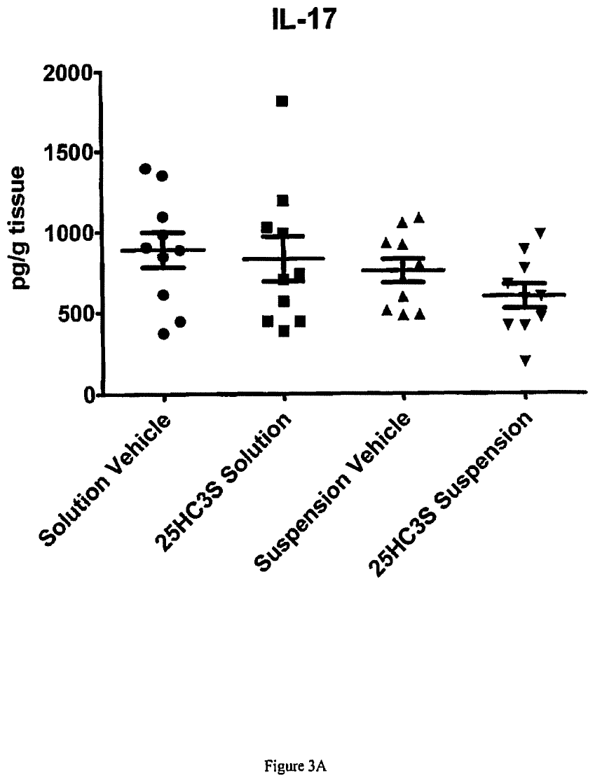

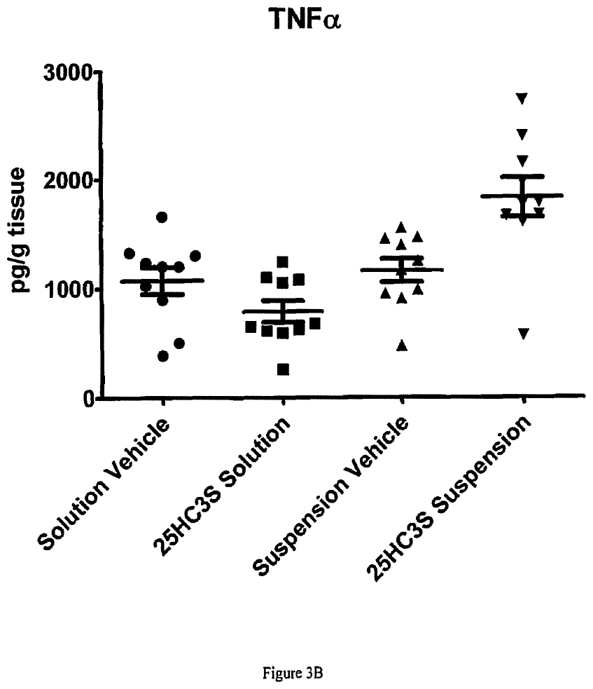

The results of this study are presented in FIGS. 2 and 3A and 3B. As can be seen in FIG. 2, erythema (redness) of the back skin was significantly reduced in mice treated with the Formulation B suspension. Erythema of the back skin was not significantly reduced in mice treated with the Formulation A, and erythema of the right ear was not significantly reduced in mice treated with Formulation A or B.

FIGS. 3A and 3B show IL-17 and TNFα protein levels, respectively, in psoriatic skin/lesions as measured by ELISA. As can be seen, IL-17 trended lower in the Formulation B group compared to the respective vehicle group whereas no major differences were observed the Formulation A and its vehicle groups. In contrast, TNFα protein levels were modestly reduced in the skin tissue of Formulation A-treated mice compared to vehicle while increased in Formulation B-treated mice compared to its respective vehicle. While these results seem contradictory, one caveat of this study is that depending on where the tissue was collected (at the site of the intradermal injection which was contained to a small region of the lesion versus unexposed regions of the psoriatic lesion), protein levels may be dramatically variable within treatment groups. In all, we find that 25HC3S promotes reduction in erythema in a rodent model of psoriasis.

### Example 3. Therapy of Chronic Dermatitis Following Poison Ivy Attack in Human

(5 mg/ml 25HC3S sodium salt in Topical Cream, External Usage) A case report: A volunteer man (60 year old) had been suffering from chronic dermatitis with intense itching following a poison ivy attack two years earlier. The affected area was externally treated with 0.5 ml of 5 mg/ml of 25HC3S sodium salt in a body lotion (Cococare®, Vitamin E Cream) once every three days for a total of three applications. Within two days, the itching subsided, and redness and swelling decreased. The skin was almost completely recovered in 10 days.

### Example 4. Topical Formulations

Topical formulations of 25HC3S were prepared using commercial vehicles and custom-made compositions.

### Evaluation of Formulations

Compositions listed were evaluated for texture, homogeneity and physical stability at room temperature, i.e., 25° C., by monitoring any sign of phase separation.

### Commercial Vehicles Used for 25HC3S Formulations for Topical Applications

PLO20™, PLO20 Flowable™, SaltStable LO™ and HRT (Hormone Replacement Therapy) Botanical Base were from HUMCO™. Vitamin E cream was from Cococare® containing 12,000 I.U. vitamin E.

### Preparation of 25HC3S in Commercial Vehicles

Formulations were prepared by addition of 25HC3S to the vehicle and mixed using a rod or homogenization. Table 1 shows the 25HC3S drug load, appearance and physical stability.

TABLE 1
Composition of formulations prepared using commercial vehicles
and vitamin E Cococare ® cream

| Formu- | | | | Physical stability | |
|---|---|---|---|---|---|
| lation | | 25HC3S | Physical | 25° C., | 1 day, |
| ID | Vehicle | % w/w | Appearance | 3 months | 32° C.* |
| 001 | HRT | 5 | Thick | Stable | No phase |
| | Base | | paste | | separation |
| 002 | Salt Stable | 5 | Thick | Stable | No phase |
| | L0 ™ | | paste | | separation |
| 003 | PLO20 ™ | 5 | Thick | Stable | Clear gel, |
| | | | paste | | cloudy when |
| | | | | | back to 25° C. |
| 004 | PLO20 | 5 | Thick | Stable | Clear gel, |
| | Flowable ™ | | paste | | cloudy when |
| | | | | | back to 25° C. |
| | | 1 | Paste | Stable | NT |
| 005 | Vitamin E | 5 | Smooth | Stable | No phase |
| | cream | | thick | | separation |
| | | | creamy | | |
| | | | paste | | |

NT: Not Tested

### Custom-Made Compositions

### Materials:

Carbopol® 971P NF and Carbopol® 974P NF were received from Lubrizol. Pluronic® F68, oleic acid, Tween® 80, Tween 60, Oleyl alcohol (Novo™), Span® 20 were received from CRODA. Lauroglcol™ 90, Transcutol®, Labrasol®, Plurol® Oleique, Labrafil® 2125cs were received from Gattefosse. DMSO was received from Gaylord Chemical Company, Dipropyl glycol from DOW Chemical Company, Lauryl lactate (Ceraphyl™ 31) from Ashland, Kolliphore® P407 (Lutrol® F127) was received from Mutcher Inc. All other additives were purchased from Spectrum.

### Preparation of Formulations:

All formulations were water based (o/w emulsions), gels and one micro emulsion. Carbopol®, Lutrol® F127, and/or Pluronic® F68 were used as thickening agents. Ethanol, Lauroglcol™ 90, Transcutol, Labrasol, Plurol® Oleique, Labrafil® 2125cs, oleic acid, HPbCD, propylene glycol (PG), Lecithin Isopropyl Palmitate Solution Base (LIPS), were used as the skin penetration enhancers. Tween® and Span® were used as surfactants. Trolamine was used to adjust pH of the formulation.

In compositions containing HPbCD (006 and 007), drug was dissolved in 25% solution of Hydroxypropyl beta cyclodextrin (HPbCD), mixed with the rest of the additives. The drug mixtures were added to the thickening agent (Carbopol®) prior to its complete gelling. All other formulations were made by adding 25HC3S powder to vehicles and mixed.

Formulations are listed in Tables 2, 4, 6 and 8. Tables 3, 5, 7 and 9 show the appearance and physical stability of the formulations. Physical stability of each formulation is shown since preparation date. Table 10 shows composition of the micro emulsion formulation and its physical stability.

TABLE 2

| Components, | Formulation ID | | | |
|---|---|---|---|---|
| % w/w | 006 | 007 | 008 | 009 |
| 25HC3S | 1.3 | 2 | 1 | 1.3 |
| Carbopol ® 971P | 1.3 | — | — | — |
| Carbopol ® 974P | — | 1 | 1 | — |
| Trolamine | 2.5 | 2 | — | — |
| Pluronic ® F68 | — | — | — | 15.2 |
| HPbCD | 6.3 | 5.5 | — | — |
| PG | 25 | 19 | — | — |
| Lauroglycol ™ 90 | — | — | — | 7.6 |
| Labrafil ® 2125cs | — | — | 7.5 | — |
| Tween ® 80 | 6.3 | 4.8 | 7.5 | 7.6 |
| Span ® 20 | — | — | 7.5 | 7.6 |
| Oleic acid | 6.3 | 4.8 | — | — |
| Methyl paraben | 0.2 | — | — | — |
| Water | 50.8 | 61 | 75.5 | 60.7 |

TABLE 3
Appearance and Physica Stability of Compositions listed in Table 2

| Formulation | Physical | Physical stability | |
|---|---|---|---|
| ID | Appearance | 25° C. | 1 day, 32° C. |
| 006 | Gel | Stable, 3 months | Vehicle: phase |
| | | | separated |
| 007 | Gel | Stable, 3 months | Phase separated, |
| | | | flows after 1 hour |
| 008 | Cream | Vehicle: phase separated | No phase |
| | | after 1.5 months | separation |
| | | Formulation: stable, | |
| | | 3 months | |
| 009 | Thick cream | Stable, 3 months | No phase |
| | | | separation |

TABLE 4

| Components, | Formulation ID | | | | |
|---|---|---|---|---|---|
| % w/w | 010 | 011 | 012 | 013 | 014 |
| 25HC3S | 5 | 1 | 1 | 5 | 5 |
| Carbopol ® 974P | 1 | 0.5 | 0.5 | 1 | 1 |
| Trolamine | 1.9 | 1 | 0.9 | 1.9 | 1.9 |
| EtOH | — | — | 9.9 | 9.5 | — |
| PG | — | — | — | — | 4.7 |
| Labrafil ® 2125cs | 9.5 | 9.9 | 9 | 9.5 | — |
| Tween ® 80 | 9.5 | 9.9 | 9 | 9.5 | 4.7 |
| Oleic acid | — | — | — | — | 4.8 |
| Methyl paraben | — | — | — | 0.2 | 0.2 |
| Water | 73.1 | 77.7 | 69.7 | 63.4 | 77.7 |

TABLE 5
Appearance and Physical Stability of Compositions listed in Table 4.

| Formulation | Physical | Physical stability | |
|---|---|---|---|
| ID | Appearance | 25° C. | 1 day, 32° C. |
| 010 | Cream | Stable, 2 months | No phase |
| | | | separation, no flow |
| 011 | Low viscosity cream | Stable, 1 month | No phase |
| | | | separation |
| 012 | Low viscosity cream | Stable, 1 month | No phase |
| | | | separation |
| 013 | Cream | Stable, 1 month | No phase |
| | | | separation, no flow |
| 014 | Thick paste | Stable, 1 month | No phase |
| | | | separation, no flow |

TABLE 6

| | Formulation ID | | | | | | | |
|---|---|---|---|---|---|---|---|---|
| Components, % w/w | 015 | 016 | 017 | 018 | 019 | 020 | 021 | 022 |
| 25HC3S | 5 | 5 | 5 | — | — | 5 | 5 | — |
| Carbopol ® 974 | — | — | 0.95 | 1 | 1 | 1 | 0.5 | 0.5 |
| LIPS | — | 19 | 19 | 20 | 20 | — | 19 | — |
| Pluronic ® F68 | 19 | 15.2 | 15.2 | 16 | — | — | 15.2 | — |
| Trolamine | — | — | 1.9 | 2 | 4 | 1.9 | 1 | 1 |
| Isopropyl Myristate | — | — | — | — | — | — | — | 10 |
| (IPM) | | | | | | | | |
| PG | — | — | — | — | — | 19 | — | — |
| ETOH | — | — | — | 6 | — | — | — | — |
| Tween ® 80 | 9.5 | — | — | — | — | — | — | 5 |
| Labrasol ® | 9.5 | — | — | — | — | — | — | — |
| Span ® 20 | 9.5 | — | — | — | — | — | — | — |
| Glyceryl monooleate | — | — | — | — | — | — | — | 10 |
| Type 40 (Peceol ™) | | | | | | | | |
| Span ® 80 | — | — | — | — | — | — | — | 5 |
| Water | 47.5 | 60.8 | 58 | 55 | 75 | 73.1 | 59.3 | 68.5 |

TABLE 7
Physical Appearance and Stability of Compositions in Table 6

| Formulation | Physical | Physical stability | |
|---|---|---|---|
| ID | Appearance | 25° C. | 1 hr, 32° C. |
| 015 | Low viscosity | Stable, | No flow, no phase |
| | gel | 1 month | separation |
| 016 | Low viscosity | Vehicle: phase | No phase |
| | emulsion | separated, 1 day | separation, |
| | | Formulation: stable, 2 | no flow |
| | | weeks | |
| 017 | Cream | Stable, 1 month | No phase |
| | | | separation, |
| | | | no flow |
| 018 | Viscous | NT | NT |
| | cream | | |
| 019 | Cream | Stable, 1 month | Stability |
| | | | questionable, |
| | | | flows |
| 020 | Clear gel | Stable, 1 month | No flow, no phase |
| | | | separation |
| 021 | Cream | Stable, 3 weeks | No flow, no phase |
| | | | separation |
| 022 | Cream | Phase separated, | NT |
| | | 1 day | |

NT: Not Tested

TABLE 8

| | Formulation ID | | | | | | |
|---|---|---|---|---|---|---|---|
| Components, % w/w | 24 | 25 | 26 | 27 | 28 | 29 | 30 |
| 25HC3S | 5 | | | 1 | 5 | | |
| Carbopol 974 | — | 1 | 1 | 1 | | 0.5 | 0.5 |
| LIPS | 19 | — | — | — | 19 | — | — |
| Lutrol F127 | 15.2 | — | — | — | 11.4 | — | — |
| Trolamine | — | 2 | 2 | 2 | | 1 | 1 |
| PEG 400 | — | 20 | — | — | — | — | — |
| PG | | | 27 | | | | |
| ETOH | — | — | 10 | — | — | — | — |
| Tween 80 | — | — | | 5 | | 5 | 5 |
| Dimethylsulfoxide | — | — | 45.5 | — | — | — | — |
| (DMSO) | | | | | | | |
| Lauryl lactate | — | — | — | 5 | — | | |
| Oleyl alcohol | — | — | — | — | — | 10 | 10 |
| Dipropylene glycol | — | — | — | — | | 10 | 10 |
| Oleic acid | — | — | — | — | — | — | 25 |
| Water | 60.8 | 77 | 14.5 | 86 | 64.6 | 73.5 | 48.5 |

TABLE 9
Physical stability of compositions listed in Table 8.

| Formulation | Physical | Physical stability | |
|---|---|---|---|
| ID | Appearance | 25° C. | 1 hr, 32° C. |
| 024 | Low viscosity | Stable, 2 weeks | No flow, no phase |
| | emulsion at 5° C. | | separation |
| | Highly viscous cream | | |
| | at 25° C. | | |
| 025 | Gel | Stable, 2 weeks | No phase |
| | | | separation, |
| | | | no flow |
| 026 | Gel | Stable, 2 weeks | No phase |
| | | | separation, |
| | | | no flow |
| 027 | Cream | Stable, 2 weeks | No phase |
| | | | separation, |
| | | | no flow |
| 028 | Thick emulsion | Stable, 1 week | NT |
| 029 | Cream | Stable, 1 week | Seems |
| | | | questionable |
| 030 | Cream | Phase separated, | |
| | | 1 day NT | |

TABLE 10
Micro Emulsion Formulation.

| Components, % w/w | Formulation ID 023 | Physical Stability at 25° C. |
|---|---|---|
| 25HC3S | 1.3 | Stable after l week |
| Transcutol ® | 7.9 | |
| Labrafil ® M 1922 CS | 4.6 | |
| Labrasol ® | 38.9 | |
| Plurol ® Oleique | 6.9 | |
| Water | 40.4 | |

### Chemical Stability of 25HC3S Topical Formulations

Chemical stability of formulations containing approximately 5% 25HC3 was monitored at 25° C. and 40° C. Samples were prepared by placing about 0.5 g formulations weighed into 2 mL glass vials, stoppered and sealed. Duplicate samples were used for each temperature and time point. Compositions used in chemical stability testing and results are listed in Table 11. Average potency of 2 samples is reported.

TABLE 11

| Formulation | Temperature, | Time, | % |
|---|---|---|---|
| ID | ° C. | weeks | 25HC3S |
| 005 | 25 | 4 | 5.1 |
| | 40 | 2 | 5.2 |
| | | 4 | 5.0 |
| 010 | 25 | 4 | 4.8 |
| | 40 | 2 | 5.0 |
| | | 4 | 4.8 |
| 013 | 25 | 4 | 4.9 |
| | 40 | 2 | 4.8 |
| | | 4 | 4.8 |
| 014 | 25 | 4 | 5.0 |
| | 40 | 2 | 5.3 |
| | | 4 | 5.0 |
| 016 | 25 | 4 | 4.6 |
| | 40 | 2 | 4.8 |
| | | 4 | 4.6 |
| 020 | 25 | 4 | 5.0 |
| | 40 | 2 | 5.2 |
| | | 4 | 4.8 |
| 021 | 25 | 4 | 4.9 |
| | 40 | 2 | 5.0 |
| | | 4 | 5.0 |

### Example 5. A Proof of Concept Study to Assess the Efficacy and Safety of Single Intralesional Doses of 25HC3S in Psoriasis Patients

### Material and Methods

The objectives of this study were:
To establish preliminary evidence for the efficacy of intralesionally injected 25HC3S in patients with psoriasis, as assessed by microplaque assay.
To assess the safety of 25HC3S in patients with psoriasis.
Compare evidence for the efficacy of different formulations of intralesionally injected 25HC3S.

### Trial Design:

This trial was a double-blind, within-participant, randomized, vehicle and active comparator-controlled, single-dose study. Participants attended a screening visit within 28 days of dosing. A target plaque(s) of psoriasis was selected.
   Day 0: each participant was treated with 2 different formulations of study drug, 2 vehicle formulations, one active comparator and one untreated area (6 treatments in total). Each treatment was administered to every participant as an intralesional injection, with the exception of the untreated area.
   Participants were required to return for outpatient visits for microplaque assessments at Day 1, Day 2, Day 7 and Day 14.
      Treatment Formulations: Table 12 lists the formulated drug products and Table 13 lists the amounts injected.

TABLE 12
Test Formulations

| Test Treatment | Formulation |
|---|---|
| 25HC3S Solution | 30 mg/mL 25HC3S in 250 mg/mL HPbCD |
| | with 10 mM sodium phosphate buffer in |
| | sterile water for injection |
| 25HC3S Suspension | 25 mg/mL 25HC3S in 3% polyethylene |
| | glycol 3350, 0.3% Polysorbate 80, 0.75% |
| | sodium chloride, 10 mM sodium phosphate |
| | buffer in sterile water for injection |
| Vehicle for Solution | 250 mg/mL HPbCD with 10 mM sodium |
| | phosphate buffer in sterile water for |
| | injection |
| Vehicle for Suspension | 3% polyethylene glycol 3350, 0.3% |
| | Polysorbate 80, 0.75% sodium chloride, 10 |
| | mM sodium phosphate buffer in sterile |
| | water for injection |
| Kenalog ®-10 | Kenalog ®-10 diluted to 2 mg/mL with |
| | 0.9% sodium chloride injection |
| Untreated area | — |

TABLE 13
Test Formulation Injections Summary

| | | Volume per | Total Delivered |
|---|---|---|---|
| | Concentration | injection (μL), | Drug/Compound |
| Test formulation | (mg/mL) | # of injections | (mg) |
| 25HC3S Solution | 30 | 100, 3 | 9 |
| 25HC3S Suspension | 25 | 100, 3 | 7.5 |
| Vehicle for Solution | — | 100, 3 | — |
| Vehicle for | — | 100, 3 | — |
| Suspension | | | |
| Kenalog ®-10 | 2 | 100, 3 | 0.5 |
| Untreated area | — | 0, 0 | — |

### Clinical Trial

Ten patients with mild to severe psoriasis were enrolled into this clinical trial after screening. For a participant to be eligible for the study, all target plaques had a Local Psoriasis Severity Index (LPSI) score ≥6. On Day 0: Each participant was treated with 2 different formulations of study drug, 2 vehicle formulations, one active comparator and one untreated area (6 treatments in total).

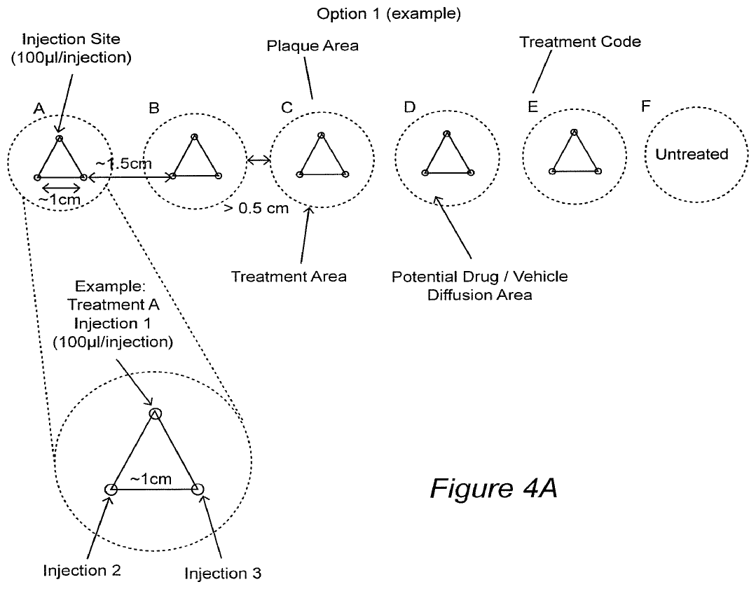

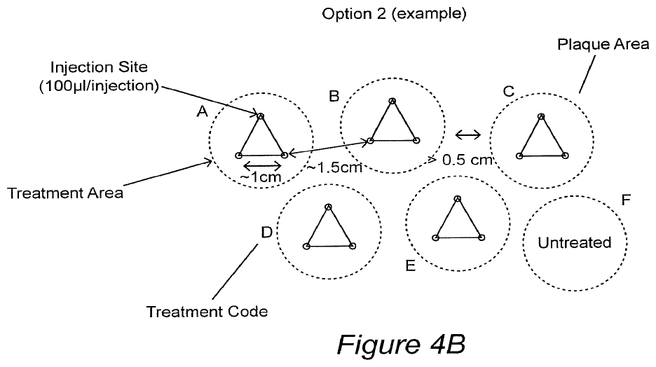

Each treatment was administered to every participant as intralesional injections to a separate small target area (microplaque) within the target plaque. Doses were administered by an unblinded injector, trained in administration of intralesional injections. Three injections of each treatment were given. The untreated areas did not receive any injections but were marked for post study observations by the unblinded injector. Diagrams of proposed injection site templates are illustrated in FIGS. 4A and B.

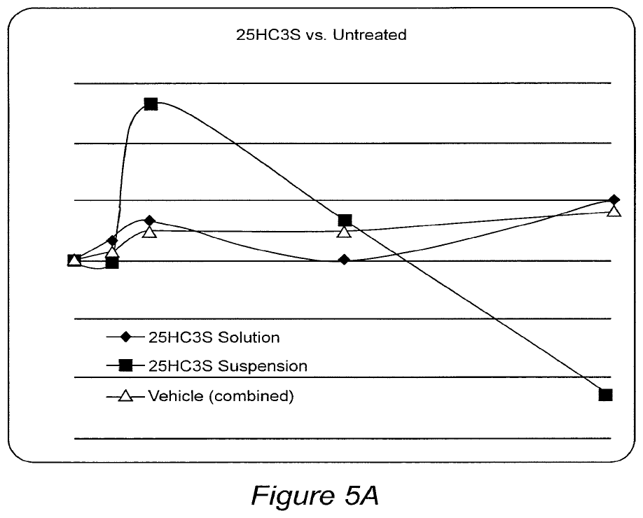

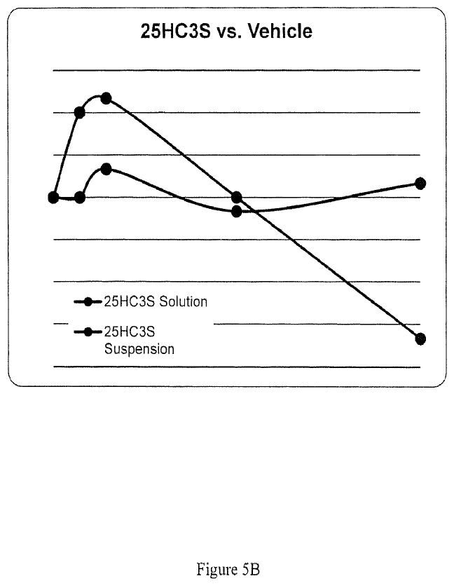

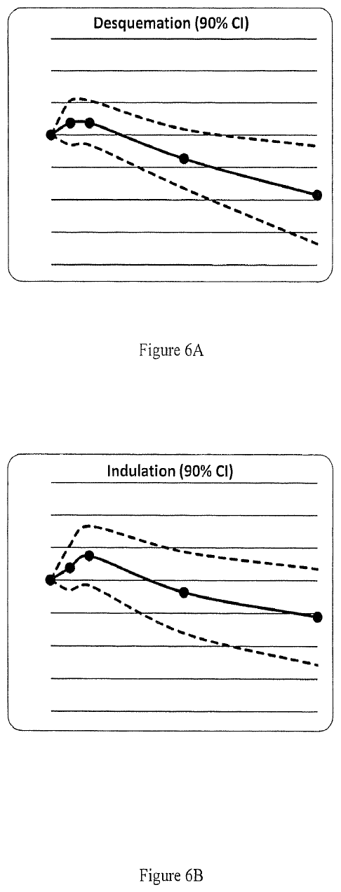

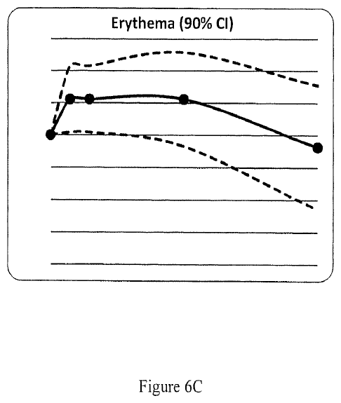

On Days 1, 2, 7 and 14: Participants were required to return for outpatient visits for microplaque assessments. The Principal Investigator graded responses to the study treatment in a blinded fashion using the LPSI, which uses a 5 point scale for scores of erythema, induration and desquamation. Results from this assessment are shown in FIGS. 5A and B and FIG. 6A-C.

### Results

The effect of 25HC3S in psoriasis was assessed by the change of LPSI score as compared to vehicle or untreated in this microplaque assay. For each formulation within a subject's target plaque, the comparison of drug vs the vehicle was made by deriving the difference and its 95% Confidence Interval (CI) of the change in LPSI scores by study visit.

As expected, a positive effect of the active comparator, Kenalog®-10, on plaques were observed (data not shown) at the conclusion of the Investigator's scoring period for this study (Day 14). 25HC3S, in a solution formulation, was not observed to have effects on ameliorating psoriasis, based on the LPSI score, compared to vehicle treatment over the 14 day scoring period (FIGS. 5A and B). In contrast, 25HC3S, in suspension, reduced the mean LPSI score by Day 14, approximately 0.7 units, compared to the vehicle (FIGS. 5A and B). An increase in LPSI of 0.8 units was also observed on Day 2, mainly attributed to a foreign body reaction from the 25HC3S particles in the suspension formulation.

A closer inspection of the categories that define the LPSI shows that 25HC3S in the suspension treatment group made the largest impact on desquamation, while decreases were also observed in indulation and erythema to lesser extents, compared to vehicle treatment (FIG. 6A-C). In conclusion, 25HC3S, given intralesionally, exhibited efficacy in psoriatic plaques by reducing LPSI in this proof of concept study.

For several patients the areas treated with a single injection of 25HC3S in suspension were observed 4 to 9 months after the injection. In at least some of these patients, the treated area appeared to have less psoriasis. In at least some of these patients, the untreated area also appeared to have less psoriasis.

### Example 6. Infusion Compatibility

25HC3S for Injection is a sterile powder, for injection solution. The 25HC3S stability with the 10 mL glass vial and FluroTec® coated stopper was studied up to 12 months at 2-8° C., 6 months at 25° C./60% RH, and 6 months at 40° C./75% RH with vials stored in the inverted orientation. Based on these stability data, it was concluded that there is good compatibility between 25HC3S and the container closure system, as shown below.

In a similar manner, the Vehicle for 25HC3S for Injection (Vehicle) stability with the 10 mL glass vial and FluroTec® coated stopper was studied up to 12 months at 2-8° C., 6 months at 25° C./60% RH, and 6 months at 40° C./75% RH with vials stored in the inverted orientation. The Vehicle was 250 mg/mL HPbCD with 10 mM phosphate buffers. Based on these stability data, it was concluded that there is good compatibility between the Vehicle and the container closure system, as shown below.

Compatibility of Constituted 25HC3S Solution with 5% Dextrose and 0.9% Sodium Chloride for Infusion and Two Kinds of Infusion Sets

After constitution with Vehicle, the 30 mg/mL 25HC3S product was diluted into 100 mL of 5% dextrose injection, USP or 0.9% sodium chloride injection, USP, and was administered to subjects as an IV infusion ranging from a 30 mg to 150 mg 25HC3S dose. This was accomplished by adding 1.0 mL (for the 30 mg dose) or 5.0 mL (for the 150 mg dose), or any volume in between, of the 30 mg/mL 25HC3S product into a 100 mL dextrose or sodium chloride infusion bag. The entire admixture content in the infusion bag was infused into the subject over approximately 2 hours at a rate of 50 mL/hour.

A physical and chemical compatibility study was conducted at a 30 mg, 48 mg and 300 mg 25HC3S dose in 5% dextrose and 0.9% sodium chloride infusion bags. Descriptions of the two infusion solutions used to dilute the constituted 25HC3S for Injection are listed in Table 14. Descriptions of the two kinds of infusion sets tested with 25HC3S product diluted in 5% dextrose and 0.9% sodium chloride are listed in Table 15. The tubing in catalog number 2H8480 infusion set was composed of polyvinylchloride (PVC), while the tubing in catalog number 2C8858 was polyethylene lined except for a short pump segment (approximately 12 inches) which was composed of PVC.

TABLE 14
Description of Infusion Solutions

| Manufacturer/Catalog | | |
|---|---|---|
| Number | Description | Size of Bag |
| Hospira | 5% Dextrose | 100 mL |
| NDC 0409-7923-23 | Injection, USP | |
| Hospira | 0.9% Sodium Chloride | 100 mL |
| NDC 0409-7984-23 | Injection, USP | |

TABLE 15
Description of Infusion Sets

| Manufacturer/ | | | |
|---|---|---|---|
| Catalog | | | |
| Number | Description | Flow Rate | Length |
| Baxter | Non-DEHP | Approximately | 103 |
| 2H8480 | Polyvinylchloride | 10 drops per | inches |
| | Solution Set with | mL | |
| | DUO-VENT spike, with | | |
| | Clearlink luer activated | | |
| | valve, with 0.22 micron filter | | |
| Baxter | Paclitaxel set with | Approximately | 107 |
| 2C8858 | polyethylene | 10 drops per | inches |
| | lined tubing, non- | mL | |
| | DEHP pump segment | | |
| | (polyvinylchloride), with | | |
| | Clearlink luer activated valve, | | |
| | with 0.22 micron filter | | |

25HC3S for Injection and Vehicle for 25HC3S for Injection, that had been stored at 2-8° C. for approximately 16 months, were used for the compatibility study. After constitution, 30 mg (1.0 mL of constituted product), 48 mg (1.6 mL of constituted product) or 300 mg (10 mL of constituted product) were added to 100 mL infusion bags of 5% dextrose and 0.9% sodium chloride, mixed thoroughly, and stored for 24 hours at room temperature and at 2-8° C. The Hospira labeled 100 mL dextrose and sodium chloride infusion bags had an overfill, so the average fill was actually 107 mL. Taking into consideration the overfill per infusion bag and the additional volume introduced by adding the constituted 25H product to each bag, the expected concentrations of 25HC3S were 0.28 mg/mL, 0.44 mg/mL, and 2.56 mg/mL in the infusion bags. Two kinds of infusion sets were then attached to the drug containing infusion bags, and the entire contents were eluted through the infusion sets at approximately 50 mL/hour at room temperature. Samples were collected from the 25H S prepared infusion bags at T=0 and at 24 hours, and from the total eluent passed through the infusion sets, and tested for 25HC3S concentration using HPLC. Solution visual appearance, osmolality (using method USP<785>), and pH (using method USP<791>) were also measured on the collected samples.

The compatibility results for 25HC3S with 5% dextrose and 0.9% sodium chloride, and with the two kinds of infusion sets, are shown in Table 16 and Table 17, respectively.

TABLE 16
Stability of 25HC3S Diluted and Stored in 5% Dextrose Infusion Bag for 24 Hours and
Eluted Through Two Kinds of Infusion Sets (Potency)

| | | | | | After Storage in Infusion |
|---|---|---|---|---|---|
| Approximate | | | 24 Hours at 25° C. | 24 Hours at 2-8° C. | Bag and Collection from |
| 25HC3S | | | Concentration in | Concentration in | Infusion Set |
| Concentration | Dextrose | T = 0 | mg/mL and | mg/mL and | Concentration in mg/mL |
| in Infusion Bag | Infusion | Conc. | (% Remaining | (% Remaining | and (% Remaining |
| (mg/mL) | Bag ID | (mg/mL) | Compared to T = 0) | Compared to T = 0) | Compared to T = 0) |
| 0.28 | 1 | 0.276 | 0.276 | | Baxter 2H8480 |
| | | | (100.0%) | | 0.276 |
| | | | | | (100.0%) |
| | 2 | 0.277 | 0.277 | | Baxter 2C8858 |
| | | | (100.0%) | | 0.277 |
| | | | | | (100.0%) |
| | 3 | 0.280 | | 0.280 | Baxter 2H8480 |
| | | | | (100.0%) | 0.280 |
| | | | | | (100.0%) |
| | 4 | 0.279 | | 0.280 | Baxter 2C8858 |
| | | | | (100.4%) | 0.280 |
| | | | | | (100.4%) |
| 0.44 | 1 | 0.447 | 0.447 | | Baxter 2H8480 |
| | | | (100.0%) | | 0.448 |
| | | | | | (100.2%) |
| | 2 | 0.442 | 0.442 | | Baxter 2C8858 |
| | | | (100.0%) | | 0.442 |
| | | | | | (100.0%) |
| | 3 | 0.439 | | 0.438 | Baxter 2H8480 |
| | | | | (99.8%) | 0.440 |
| | | | | | (100.2%) |
| | 4 | 0.439 | | 0.440 | Baxter 2C8858 |
| | | | | (100.2%) | 0.440 |
| | | | | | (100.2%) |
| 2.56 | 1 | 2.500 | 2.470 | | Baxter 2H8480 |
| | | | (98.8%) | | 2.480 |
| | | | | | (99.2%) |
| | 2 | 2.510 | 2.520 | | Baxter 2C8858 |
| | | | (100.4%) | | 2.545 |
| | | | | | (101.4%) |
| | | | | 2.550 | Baxter 2H8480 |
| | 3 | 2.530 | | (100.8%) | 2.540 |
| | | | | | (100.4%) |
| | 4 | 2.525 | | 2.530 | Baxter 2C8858 |
| | | | | (100.2%) | 2.535 |
| | | | | | (100.4%) |

TABLE 17
Stability of 25HC3S Diluted and Stored in 0.9% Sodium Chloride Infusion Bag for 24
Hours and Eluted Through Two Kinds of Infusion Sets (Potency)

| | | | | | After Storage in Infusion |
|---|---|---|---|---|---|
| Approximate | | | 24 Hours at 25° C. | 24 Hours at 2-8° C. | Bag and Collection from |
| 25HC35 | Sodium | | Concentration in | Concentration in | Infusion Set |
| Concentration | Chloride | T = 0 | (mg/mL) and | mg/mL and | Concentration in mg/mL |
| in Infusion Bag | Infusion | Conc. | (% Remaining | (% Remaining | and (% Remaining |
| (mg/mL) | Bag ID | (mg/mL) | Compared to T = 0) | Compared to T = 0) | Compared to T = 0) |
| 0.28 | 1 | 0.273 | 0.272 | | Baxter 2H8480 |
| | | | (99.6%) | | 0.272 |
| | | | | | (99.6%) |
| | 2 | 0.274 | 0.274 | | Baxter 2C8858 |
| | | | (100%) | | 0.274 |
| | | | | | (100%) |
| | 3 | 0.274 | | 0.275 | Baxter 2H8480 |
| | | | | (100.4%) | 0.275 |
| | | | | | (100.4%) |
| | 4 | 0.275 | | 0.275 | Baxter 2C8858 |
| | | | | (100.0%) | 0.275 |
| | | | | | (100.0%) |
| 0.44 | 1 | 0.434 | 0.434 | | Baxter 2H8480 |
| | | | (100.0%) | | 0.433 |
| | | | | | (99.8%) |
| | 2 | 0.424 | 0.424 | | Baxter 2C8858 |
| | | | (100.0%) | | 0.424 |
| | | | | | (100.0%) |
| | 3 | 0.434 | | 0.434 | Baxter 2H8480 |
| | | | | (100.0%) | 0.434 |
| | | | | | (100.0%) |
| | 4 | 0.425 | | 0.425 | Baxter 2C8858 |
| | | | | (100.0%) | 0.426 |
| | | | | | (100.2%) |
| 2.56 | 1 | 2.450 | 2.500 | | Baxter 2H8480 |
| | | | (102.0%) | | 2.460 |
| | | | | | (100.4%) |
| | 2 | 2.530 | 2.525 | | Baxter 2C8858 |
| | | | (99.8%) | | 2.520 |
| | | | | | (99.6%) |
| | 3 | 2.530 | | 2.510 | Baxter 2H8480 |
| | | | | (99.2%) | 2.530 |
| | | | | | (100.0%) |
| | 4 | 2.525 | | 2.520 | Baxter 2C8858 |
| | | | | (99.8%) | 2.520 |
| | | | | | (99.8%) |

The 25HC3S concentrations in 5% dextrose after 24 hours at room temperature and at 2-8° C., and after elution through the infusion sets were all within 1.4% of the target concentrations of the initial T=0 time point. Similar 25HC3S stability in 0.9% sodium chloride was observed, where after 24 hours at room temperature and at 2-8° C., and after elution through the infusion sets all the concentrations were within 2.0% of the target concentrations of the initial T=0 time point.

Osmolality and pH data for 25HC3S in 5% dextrose at T=0 and 24 hours, and after elution through the two kinds of infusion sets are shown in Table 18. Osmolality and pH data for 25HC3S in 0.9% sodium chloride at T=0 and 24 hours, and after elution through two kinds of infusion sets are shown in Table 19. The osmolality data, for both the dextrose and sodium chloride drug containing solutions, showed no consistent trends over time in the infusion bag or after elution through the infusion sets. The pH of the dextrose drug containing solutions also showed no trends over time or after elution through the infusion sets. The pH of the sodium chloride drug containing solution at approximately 0.28 mg/mL 25HC3S showed an approximate decrease of 0.5 of a pH unit over 24 hours in the infusion bags, and appeared to decrease by approximately a tenth of a pH after elution through the infusion sets. The pH of the sodium chloride drug containing solution at approximately 0.44 mg/mL 25HC3S showed no consistent trends over time in the infusion bags, but appeared to decrease by a few tenths of a pH after elution through the infusion sets. The pH of the sodium chloride drug containing solution at approximately 2.56 mg/mL 25HC3S showed a slight decrease by a tenth of a pH over time in the infusion bags, and appeared to drop by a few tenths of a pH after elution through the infusion sets.

The 25HC3S solutions in dextrose and sodium chloride, at all three concentrations, remained as clear and colorless solutions, after 24 hours in the infusion bags, and after elution through the infusion sets.

The appearance of the infusion bags and infusions sets also remained the same before and after use with the 25HC3S solutions.

The compatibility of 25HC3, at 30 mg, 48 mg, and 300 mg, as admixtures with 100 mL of dextrose and sodium chloride, and with two kinds of infusion sets, has been demonstrated by the acceptable 25HC3S concentration, pH, osmolality, and physical appearance stability data.

TABLE 18
Stability of 25HC3S Diluted and Stored in 5% Dextrose Infusion Bag for 24 Hours and
Eluted Through Two Kinds of Infusion Sets (Osmolality and pH)

| Approximate | | | | | After Storage in Infusion |
|---|---|---|---|---|---|
| 25HC3S | | T = 0 | | | Bag and Collection from |
| Concentration | Dextrose | Osmolality | 24 Hours at 25° C. | 24 Hours at 2-8° C. | Infusion Set |
| in Infusion Bag | Infusion | (mmol/kg) | Osmolality | Osmolality | Osmolality (mmol/kg) |
| (mg/mL) | Bag ID | and pH | (mmol/kg) and pH | (mmol/kg) and pH | and pH |
| 0.28 | 1 | 247 | 252 | | Baxter 2H8480 |
| | | 7.09 | 7.12 | | 252 |
| | | | | | 6.99 |
| | 2 | 250 | 249 | | Baxter 2C8858 |
| | | 7.03 | 7.05 | | 252 |
| | | | | | 7.04 |
| | 3 | 251 | | 250 | Baxter 2H8480 |
| | | 7.10 | | 7.09 | 251 |
| | | | | | 6.95 |
| | 4 | 250 | | 253 | Baxter 2C8858 |
| | | 7.04 | | 7.04 | 252 |
| | | | | | 6.99 |
| 0.44 | 1 | 255 | 256 | | Baxter 2H8480 |
| | | 6.83 | 6.91 | | 253 |
| | | | | | 6.91 |
| | 2 | 254 | 253 | | Baxter 2C8858 |
| | | 6.81 | 6.90 | | 253 |
| | | | | | 6.90 |
| | 3 | 256 | | 245 | Baxter 2H8480 |
| | | 6.82 | | 6.96 | 257 |
| | | | | | 6.91 |
| | 4 | 255 | | 257 | Baxter 2C8858 |
| | | 6.88 | | 6.93 | 255 |
| | | | | | 6.93 |
| 2.56 | 1 | 258 | 257 | | Baxter 2H8480 |
| | | 6.04 | 6.12 | | 261 |
| | | | | | 6.01 |
| | 2 | 247 | 250 | | Baxter 2C8858 |
| | | 6.12 | 6.06 | | 251 |
| | | | | | 6.00 |
| | 3 | 247 | | 246 | Baxter 2H8480 |
| | | 6.29 | | 6.11 | 247 |
| | | | | | 5.99 |
| | 4 | 247 | | 251 | Baxter 2C8858 |
| | | 6.26 | | 6.14 | 248 |
| | | | | | 6.10 |

TABLE 19
Stability of 25HC3S Diluted and Stored in 0.9% Sodium Chloride Infusion Bag for 24
Hours and Eluted Through Two Kinds of Infusion Sets (Osmolality and pH)

| Approximate | | | | | After Storage in Infusion |
|---|---|---|---|---|---|
| 25HC3S | Sodium | T = 0 | | | Bag and Collection from |
| Concentration | Chloride | Osmolality | 24 Hours at 25° C. | 24 Hours at 2-8° C. | Infusion Set |
| in Infusion Bag | Infusion | (mmol/kg) | Osmolality | Osmolality | Osmolality (mmol/kg) |
| (mg/mL) | Bag ID | and pH | (mmol/kg) and pH | (mmol/kg) and pH | and pH |
| 0.28 | 1 | 278 | 275 | | Baxter 2H8480 |
| | | 6.39 | 5.93 | | 276 |
| | | | | | 5.78 |
| | 2 | 277 | 277 | | Baxter 2C8858 |
| | | 6.43 | 5.92 | | 276 |
| | | | | | 5.83 |
| | 3 | 277 | | 276 | Baxter 2H8480 |
| | | 6.45 | | 5.87 | 277 |
| | | | | | 5.81 |
| | 4 | 278 | | 276 | Baxter 2C8858 |
| | | 6.40 | | 5.99 | 276 |
| | | | | | 5.78 |
| 0.44 | 1 | 277 | 280 | | Baxter 2H8480 |
| | | 7.30 | 7.33 | | 286 |
| | | | | | 7.09 |
| | 2 | 279 | 280 | | Baxter 2C8858 |
| | | 7.43 | 7.41 | | 288 |
| | | | | | 7.20 |
| | 3 | 284 | | 281 | Baxter 2H8480 |
| | | 7.21 | | 7.43 | 282 |
| | | | | | 7.13 |
| | 4 | 280 | | 281 | Baxter 2C8858 |
| | | 7.44 | | 7.19 | 281 |
| | | | | | 7.16 |
| 2.56 | 1 | 282 | 282 | | Baxter 2H8480 |
| | | 6.20 | 6.01 | | 283 |
| | | | | | 5.81 |
| | 2 | 282 | 282 | | Baxter 2C8858 |
| | | 6.10 | 5.86 | | 279 |
| | | | | | 5.83 |
| | 3 | 282 | | 283 | Baxter 2H8858 |
| | | 6.07 | | 5.93 | 283 |
| | | | | | 5.72 |
| | 4 | 281 | | 283 | Baxter 2C8858 |
| | | 6.11 | | 5.96 | 283 |
| | | | | | 5.70 |

### Example 7. Formulation Physical Stability Testing

### Methods

The formulations shown in below Tables 20 and 21 were made as follows. The 25HC3S was dissolved in a mixture of solvents/penetration enhancers/surfactant excluding water. Carbopol® polymer was separately dissolved in water and trolamine was added to form a gel. The solution of 25HC3S was then added to the Carbopol gel and mixed. The final formulations were typically a cream or gel.

### Results

The appearance of the resulting formulations is shown in below Tables 20 and 21. Most of the formulations were left at room temperature for 4 months. Their physical stability was recorded as shown in below Tables 20 and 21.

TABLE 20
Formulations for Physical Stability Studies

| Components, % w/w | Form ID 31 | Form ID 32 | Form ID 33 | Form ID 34 | Form ID 35 | Form ID 36 |
|---|---|---|---|---|---|---|
| 25HC3S | 1 | 1 | 1 | 1 | 1 | 1 |
| Carbopol 974P | 0.5 | 0.5 | 1 | 1 | 1 | 1 |
| Trolamine | 1 | 1 | 2 | 2 | 2 | 2 |
| Propylene Glycol | 24.8 | 39.6 | 39.6 | 39.6 | 14.8 | — |
| PEG 400 | — | — | — | — | — | 39.6 |
| Oleic acid | 24.8 | — | — | — | 9.9 | — |
| Tween 80 | 9.9 | 9.9 | 9.9 | | 9.9 | — |
| Water | 38 | 48 | 46.5 | 53.4 | 61.4 | 56.4 |
| Appearance | Cream | Cloudy | Low | Gel | Cream | Gel |
| | | solution | viscosity | | | |
| | | | gel | | | |
| Physical stability | Phase | Stable | Stable | Stable | Stable | Stable |
| at room temperature, | separated | | | | | |
| 4 months | after 1 day | | | | | |

TABLE 21
Formulations for Physical Stability Studies

| Components, | Form | Form | Form | Form | Form |
|---|---|---|---|---|---|
| % w/w | ID 37 | ID 38 | ID 39 | ID 41 | ID 41-1 |
| 25HC3S | 1 | 1 | 1 | 1 | 1 |
| Carbopol 974P | 1 | 0.5 | 1 | 1 | — |
| Trolamine | 2 | 1 | 2 | 2 | — |
| PEG 400 | — | — | — | 44.5 | 34.6 |
| Propylene | 19.8 | — | — | — | — |
| Glycol | | | | | |
| Di PG | — | — | 19.8 | — | — |
| Oleyl alcohol | — | — | 9.9 | — | — |
| ETOH | 9.9 | — | — | — | — |
| DMSO | 19.8 | — | — | 19.8 | 9.9 |
| Lauryl lactate | — | 19.8 | — | | |
| LIPS | — | — | — | 14.8 | 14.8 |
| Lutrol F127 | | | | | 7.9 |
| Tween 80 | — | 9.9 | 9.9 | 4.9 | — |
| Water | 46.5 | 67.8 | 56.4 | 11.9 | 31.7 |
| Appearance | Clear | Cream | Cream | Biphasic | Gel |
| | gel | | | Mixture | |
| Physical stability | Stable | Not | Stable | Did not | Phase |
| at room | | tested | | form | separated |
| temperature, 4 | | | | cream | |
| months | | | | | |

### Example 8. First Cadaver Skin Study

### Objectives

Increase drug content permeated in skin
Improve stability of topical formulations
Increase drug solubility in formulations

### Strategy

Commercial vehicles
   Vitamin E cream from Cococare
   Four other vehicles
Vehicles developed and evaluated in house (focusing on cream or gel)
   All vehicles were water based (W/O emulsion and gels)
   Thickening agents: Carbopol 974 (crosslinked polyacrylic acid polymer), Pluronic F68
   Emulsifiers: Tween 80, Span 20
   Skin permeation enhancers
      EtOH, Propylene glycol, DiPG, lauryl lactate, oleic acid, oleyl alcohol, lipidic excipients (labrafil M2125, Lauroglycol), lecithin isopropyl palmitate solution (LIPS)

### Methods

The formulations shown in the below Table 22 were made. Each of the below formulations containing drug included 1 wt % non-radiolabelled 25HC3S since the maximum drug loading achieved in Carbopol based creams or gels was 1 wt %. In positive control C1, 20 wt % DMSO was included in EtOH to achieve 1 wt % drug loading.

The procedure for mixing radiolabelled C¹⁴-25HC3S with each formulation was as follows. Formulation (1 mL of each) was placed into 1 mL vials. To each vial was added 5 EtOH containing hot material (C¹⁴ radiolabelled 25HC3S). The mixture was mixed using a small plunger for 4 to 5 minutes until uniform.

TABLE 22
Topical Formulations Containing 1% 25HC3S in 1^{st} Cadaver Skin Flux Study

| | | | | | | | | | | Positive | Negative |
|---|---|---|---|---|---|---|---|---|---|---|---|
| Components, % | F1 | F2 | F3 | F4 | F5 | F6 | F7 | F8 | F9 | Control C1 | Control C2 |
| 25HC3S | Vitamin | 1 | 1 | 1 | 1 | 1 | 1 | 1 | 1 | 1 | 1 |
| Carbopol 974 | E | 1 | 1 | 1 | 1 | 1 | 1 | 1 | 1 | — | 1 |
| Trolamine | Cococare | 2 | 2 | 2 | 2 | 1.9 | 2 | 1.9 | 2 | — | 2 |
| Lauryl lactate | | — | — | 4.95 | — | — | — | — | — | — | — |
| PG | | — | — | | 39.6 | 14.9 | — | 19.8 | — | — | — |
| PEG 400 | | — | — | — | — | — | 39.6 | — | — | — | — |
| ETOH | | — | 9.9 | — | — | — | — | 10 | — | 79.2 | — |
| DMSO | | — | — | — | — | — | — | 19.8 | — | 19.8 | — |
| Oleic acid | | — | — | — | — | 9.9 | — | — | — | — | — |
| Tween 80 | | 9.9 | 9.9 | 4.95 | 3 | 9.9 | — | — | 9.9 | — | — |
| Oleyl alcohol | | — | — | — | — | — | — | — | 9.9 | — | — |
| Di | | — | — | — | — | — | — | — | 19.8 | — | — |
| propylene | | | | | | | | | | | |
| glycol | | | | | | | | | | | |
| Labrafil | | 9.9 | 9.9 | — | — | — | — | — | — | — | — |
| M2125 | | | | | | | | | | | |
| Water | | 76.2 | 66.3 | 86.1 | 53.4 | 61.4 | 56.4 | 46.5 | 56.43 | | 96.03 |

The above formulations were tested on cadaver skin as follows. Dermatomed cadaver skin was obtained from thigh and abdominal areas. A total of 4 donor skin samples (4 separate experiments) were used in the study. Skin samples were placed on diffusion cells (see below) at least 2 hours prior to dosing. Skin sample integrity was examined by measuring total epidermal water loss (TEWL).

The diffusion cells had 1 cm² surface area. Each sample at each testing point had 2-3 replicates.

The dose was 10-25 μL of formulation each containing 0.2-0.5 μCi radioactivity per diffusion cell.

The receptor fluid was 6% PEG 400 in PBS. The receptor fluid flow rate was continuous flow at 4.7 mL/hr.

For dose application, the net amount was determined by weight difference before and after dosing application.

The total skin exposure time was 24 hours. After 8 hours of skin exposure, skin surface dose residues were removed by 5% soap-water washing as follows: (1) two times with small cotton balls wetted with 5% clear Ivory® liquid soap (Proctor and Gamble); and (2) two times with cotton balls wetted with distilled de-ionized water to recover the residual drug content, and a final drying with a dry cotton ball. After 16 hours of additional skin exposure (after skin washing), the experiment was finished.

The dosed skin was first tape stripped 10 times followed with heat separation of viable epidermis and dermis. Receptor fluid samples were collected at 30 min, 1 hour, 2 hour, and every 2 hours until the end of the experiment. All samples were counted for radioactivity

### Results

As noted above the study involved 4 skin donors with 3 permeation experiments per donor. Very trace amount of drug was found in the receptor fluid. The results are shown in below Tables 23 and 24.

TABLE 23

| | % Dose recovered | | | | | | | | | | | |
|---|---|---|---|---|---|---|---|---|---|---|---|---|
| | F1 | | F2 | | F3 | | F4 | | F5 | | F6 | |
| Sample Items | Mean | SD | Mean | SD | Mean | SD | Mean | SD | Mean | SD | Mean | SD |
| Tape strips 1-2 | 1.07 | 0.67 | 1.27 | 1.57 | 4.26 | 6.42 | 2.07 | 2.21 | 3.29 | 2.48 | 4.29 | 2.04 |
| Tape strips 3-4 | 0.37 | 0.24 | 0.43 | 0.41 | 1.79 | 2.65 | 0.71 | 0.80 | 1.30 | 1.29 | 1.76 | 0.84 |
| Tape strips 5-6 | 0.26 | 0.22 | 0.23 | 0.23 | 0.52 | 0.42 | 0.49 | 0.60 | 0.76 | 0.51 | 0.97 | 0.39 |
| Tape strips 7-8 | 0.09 | 0.08 | 0.17 | 0.19 | 0.27 | 0.16 | 0.21 | 0.18 | 0.64 | 0.61 | 0.79 | 0.55 |
| Tape strips 9-10 | 0.07 | 0.05 | 0.12 | 0.12 | 0.26 | 0.13 | 0.19 | 0.18 | 0.42 | 0.38 | 0.44 | 0.17 |
| Epidermis | 0.41 | 0.35 | 0.41 | 0.58 | 0.57 | 0.47 | 0.88 | 0.97 | 1.52 | 0.97 | 1.23 | 0.78 |
| Dermis | 0.11 | 0.08 | 0.13 | 0.12 | 0.16 | 0.11 | 0.10 | 0.14 | 0.14 | 0.11 | 0.24 | 0.12 |
| Edge (non-dosed) | 0.39 | 0.31 | 0.44 | 0.33 | 1.52 | 2.18 | 0.33 | 0.34 | 1.03 | 1.14 | 2.09 | 2.58 |
| Surface wash | 97.28 | 7.32 | 91.72 | 5.73 | 82.69 | 13.60 | 91.02 | 7.30 | 96.96 | 9.42 | 86.55 | 11.59 |
| Surface unabsorbed | 98.35 | 7.13 | 92.99 | 4.75 | 89.64 | 10.41 | 93.09 | 5.95 | 100.25 | 7.85 | 93.69 | 10.26 |
| Stratum corneum | 0.80 | 0.46 | 0.96 | 0.92 | 2.85 | 3.18 | 1.61 | 1.61 | 3.13 | 2.72 | 3.97 | 1.78 |
| Deep skin | 0.53 | 0.40 | 0.54 | 0.69 | 0.74 | 0.51 | 0.99 | 0.96 | 1.67 | 1.02 | 1.47 | 0.85 |
| In dosed skin | 1.32 | 0.81 | 1.50 | 1.45 | 3.59 | 3.56 | 2.60 | 2.37 | 4.80 | 3.66 | 5.44 | 2.28 |
| Undosed skin | 0.39 | 0.31 | 0.44 | 0.33 | 1.52 | 2.18 | 0.33 | 0.34 | 1.03 | 1.14 | 2.09 | 2.58 |
| Total skin absorbed | 1.72 | 0.97 | 1.95 | 1.76 | 5.11 | 3.80 | 2.94 | 2.45 | 5.83 | 4.54 | 7.54 | 2.90 |

TABLE 24

| | % Dose recovered | | | | | | | | | |
|---|---|---|---|---|---|---|---|---|---|---|
| | F7 | | F8 | | F9 | | Control 1 | | Control 2 | |
| Sample Items | Mean | SD | Mean | SD | Mean | SD | Mean | SD | Mean | SD |
| Tape strips 1-2 | 2.60 | 2.85 | 4.44 | 5.32 | 4.74 | 3.43 | 10.88 | 8.73 | 1.47 | 1.55 |
| Tape strips 3-4 | 1.07 | 1.34 | 0.91 | 0.57 | 1.74 | 1.52 | 3.75 | 2.33 | 0.29 | 0.30 |
| Tape strips 5-6 | 0.71 | 0.89 | 0.63 | 0.57 | 1.13 | 0.82 | 2.04 | 1.46 | 0.28 | 0.36 |
| Tape strips 7-8 | 0.33 | 0.39 | 0.41 | 0.37 | 0.85 | 0.68 | 1.30 | 0.72 | 0.17 | 0.22 |
| Tape strips 9-10 | 0.23 | 0.23 | 0.22 | 0.11 | 0.70 | 0.49 | 0.62 | 0.38 | 0.11 | 0.15 |
| Epidermis | 0.70 | 0.59 | 1.04 | 0.80 | 1.26 | 0.79 | 2.83 | 1.48 | 0.38 | 0.45 |
| Dermis | 0.19 | 0.23 | 0.20 | 0.22 | 0.72 | 0.53 | 0.58 | 0.53 | 0.09 | 0.07 |
| Edge (nondosed) | 1.26 | 2.33 | 0.54 | 0.43 | 5.81 | 5.51 | 1.29 | 0.96 | 0.69 | 1.18 |
| Surface wash | 96.06 | 11.79 | 90.45 | 12.55 | 67.36 | 18.12 | 65.75 | 25.52 | 100.35 | 14.80 |
| Surface unabsorbed | 98.66 | 10.02 | 94.90 | 7.74 | 72.11 | 17.34 | | | 101.82 | 15.35 |
| Stratum corneum | 2.36 | 2.75 | 2.18 | 1.50 | 4.44 | 3.40 | 7.72 | 4.25 | 0.87 | 0.96 |
| Deep skin | 0.89 | 0.76 | 1.24 | 0.93 | 1.98 | 0.79 | 3.41 | 1.81 | 0.47 | 0.49 |
| In dosed skin | 3.25 | 3.34 | 3.43 | 2.29 | 6.41 | 3.92 | 11.13 | 5.71 | 1.34 | 1.45 |
| Undosed skin | 1.26 | 2.33 | 0.54 | 0.43 | 5.81 | 5.51 | 1.29 | 0.96 | 0.69 | 1.18 |
| Total skin absorbed | 4.51 | 5.25 | 3.97 | 2.65 | 12.23 | 5.71 | 12.43 | 6.13 | 2.03 | 2.25 |

All in-house formulations (except F2) were better than the commercial vehicle (F1), based on amount of drug in deep skin. The order of results was C1>F9>F5>F6>F8>F4>F7.
C1: EtOH (80%), DMSO (20%)
F9: oleyl alcohol (10%), diPG (20%), H₂O (57%)
F5: PG (40%), H₂O (54%)
F6: PG (15%), oleic acid (10%), H₂O (62%)
F8: PG (20%), EtOH (10%), DMSO (20%), H₂O (47%)
F4: Lauryl lactate (2.5%), H₂O (87%)
F7: PEG400 (40%), H₂O (57%)

The following trends were seen for Permeation Enhancers (PE)
C1 vs. F8
   EtOH: high PE
F9 vs. F5
   OAlc+diDP better than PG
F5 vs. F7
   PG better than PEG400
F5 vs. F6
   PG may be about the same as OA.
F5 vs. F4
   LL may be more effective than PG (diffusion per concentration unit).

### Example 9. Formulation Chemical Stability Testing

Formulations F4, F5, F6, and F9 from Example 8 were tested for chemical stability as shown in Table 25. After the formulations were stored for 3 weeks at the temperature shown below, the amount of drug remaining was assayed by HPLC.

TABLE 25
Chemical stability of some formulations used in Example 8

| | | | % |
|---|---|---|---|
| | | | Remained |
| Formulation | Time, | | Based on |
| ID | weeks | Temperature, ° C. | 5° C. |
| F4 | 3 | 5 | — |
| | | 25 | 99.6 |
| | | 40 | 99.5 |
| F5 | 3 | 5 | — |
| | | 25 | 100.2 |
| | | 40 | 100.0 |
| F6 | 3 | 5 | — |
| | | 25 | 100.0 |
| | | 40 | 101.0 |
| F9 | 3 | 5 | — |
| | | 25 | 99.2 |
| | | 40 | 100.5 |

### Example 10. Formulation Physical Stability Testing

### Methods

The formulations shown in below Tables 26 and 27 were made using the procedure described in Example 7, except for Formulations 44, 46, 48, and 50. The final formulations were typically a cream or gel.

Formulation 44 was prepared by following the below steps:
1) Drug was dissolved in a solution of HPbCD in water.
2) Isopropyl palmitate and Tween 60 were mixed with molten cetyl alcohol at 60° C.
3) Drug solution was added to the mixture of cetyl alcohol/IPM and Tween 60 and mixed until a uniform cream was formed.

Formulations 46, 48, and 50 were prepared by following the below steps:
1) Drug was dissolved in mixture of solvents/penetration enhancers.
2) Silicon dioxide was then added and mixed until gel was formed.

### Results

The appearance of the resulting formulations is shown in below Tables 26 and 27. Some of the formulations were left at room temperature for 2 months. Their physical stability was recorded as shown in below Table 26.

TABLE 26
Formulations for Physical Stability Studies

| Components, | Form | Form | Form | Form |
|---|---|---|---|---|
| % w/w | ID 42 | ID 43 | ID 44 | ID 45 |
| 25HC3S | — | 1 | 1 | — |
| Carbopol 974P | 1 | 1 | — | — |
| Trolamine | 2 | 2 | — | |
| Hydroxypropyl | — | — | — | 3 |
| cellulose | | | | |
| HPbCD | — | 5.9 | 5.9 | — |
| IPM | — | 46.5 | 39.6 | — |
| Cetyl alcohol | — | — | 9.9 | — |
| ETOH | 40 | — | — | 26 |
| DMSO | 10 | — | — | 45.5 |
| Propylene Glycol | — | — | — | 11 |
| Tween 60 | — | 9.9 | 9.9 | — |
| Water | 47 | 33.7 | 33.7 | 14.5 |
| Appearance | Gel | Cream | Cream | Clear |
| | | | | thin gel |
| Physical stability | Stable | Stable | Stable cream | Not |
| at room | after | after | after | tested |
| temperature | 3 months | 2 months | 2 months | |
| | | | (cetyl alcohol | |
| | | | solidified) | |

TABLE 27
Formulations for Physical Stability Studies

| Components, | Form | Form | Form | Form |
|---|---|---|---|---|
| % w/w | ID 46 | ID 47 | ID 48 | ID 50 |
| 25HC3S | 9 | 8.4 | 5.7 | 5.5 |
| PEG 400 | 83.7 | 64.1 | 3.6 | 3.1 |
| ETOH | — | — | 9.7 | 8.5 |
| DMSO | — | — | — | — |
| Propylene Glycol | — | 27.5 | — | 39 |
| DiPG | — | — | 50.7 | — |
| Oleyl alcohol | — | — | 25.4 | — |
| Oleic acid | — | — | — | 39 |
| Lauryl lactate | | | | |
| Water | | | — | — |
| Silicon dioxide, | 7.3 | | 5 | 5 |
| SiO2 | | | | |
| Appearance | Opaque | Solution | Low | Thin |
| | Gel | | viscosity | gel |
| | | | hazy gel | |
| Physical stability | Not | Not | Not tested | Not |
| at room | tested | tested | | tested |
| temperature | | | | |

### Example 11. Second Cadaver Skin Study

### Objectives

Maximize drug content permeated in skin
Based on F9 and F5/F6
Increase permeation capability
Maximize drug loading

### Strategy

Use multiple permeation enhancers for synergistic effect
Reduce water to increase drug solubility (do not use Carbopol as thickening agent)
Increase PG: good permeation enhancer and fair solubilizer (˜30 mg/mL)
Add/keep EtOH: great permeation enhancer but not so good solubilizer (˜3 mg/mL)
Add a small amount of PEG 400: poor permeation enhancer but great solubilizer (˜130 mg/mL)
Use SiO₂ as thickening agent which does not require water

### Methods

The formulations shown in the below Table 28 were made by using the procedure described in Example 8.

TABLE 28
Formulations used in the 2^{nd} cadaver skin flux test

| Components, | | | | | Positive Control |
|---|---|---|---|---|---|
| % | F11 | F12 | F13 | F14 | C1 |
| 25HC3S | 1 | 6 | 1 | 6 | 1 |
| Lauryl Lactate | 2.5 | 2.35 | 2.5 | 2.35 | |
| PG | 44.5 | 42.3 | 64.3 | 61.1 | |
| PEG 400 | 4.9 | 4.7 | 4.9 | 4.7 | |
| ETOH | 10 | 9.4 | 10 | 9.4 | 79.2 |
| DMSO | — | — | — | — | 19.8 |
| Oleic Acid | — | — | 9.9 | 9.4 | |
| Oleyl Alcohol | 9.9 | 9.4 | — | — | |
| Di propylene | 19.8 | 18.8 | — | — | |
| Glycol | | | | | |
| SiO₂ | 4.9 | 4.7 | 4.9 | 4.7 | |
| Water | 2.5 | 2.35 | 2.5 | 2.35 | |

### Results

The study involved one skin donor with 5 permeation experiments. Very trace amount of drug was found in the receptor fluid. The results are shown in below Table 29.

TABLE 29

| | One Donor | | One Donor | | One Donor | | One Donor | | One Donor | |
|---|---|---|---|---|---|---|---|---|---|---|
| | F11 (1% DL) | | F12 (6% DL) | | F13 (1% DL) | | F14 (6% DL) | | FC1 (1% DL) | |
| | Mean | SD | Mean | SD | Mean | SD | Mean | SD | Mean | SD |
| Surface unabsorbed | 92.72 | 3.65 | 96.28 | 5.07 | 86.16 | 13.29 | 90.25 | 6.99 | 68.67 | 9.12 |
| Stratum corneum | 0.60 | 0.36 | 0.35 | 0.11 | 0.79 | 0.37 | 0.34 | 0.28 | 3.57 | 1.71 |
| Deep skin | 1.31 | 0.44 | 0.53 | 0.09 | 1.03 | 1.04 | 0.77 | 0.76 | 8.32 | 7.78 |
| In dosed skin | 1.92 | 0.81 | 0.90 | 0.13 | 1.83 | 1.34 | 1.12 | 0.86 | 11.90 | 9.36 |
| Undosed skin | 0.38 | 0.16 | 0.12 | 0.01 | 0.86 | 0.82 | 0.08 | 0.03 | 2.88 | 0.80 |
| Total skin absorbed | 2.31 | 0.94 | 1.03 | 0.13 | 2.69 | 2.07 | 1.21 | 0.89 | 14.79 | 10.16 |
| Amount of Drug | 1.97 | | 4.81 | | 1.54 | | 6.92 | | 12.47 | |
| (μg) Deep Skin | | | | | | | | | | |

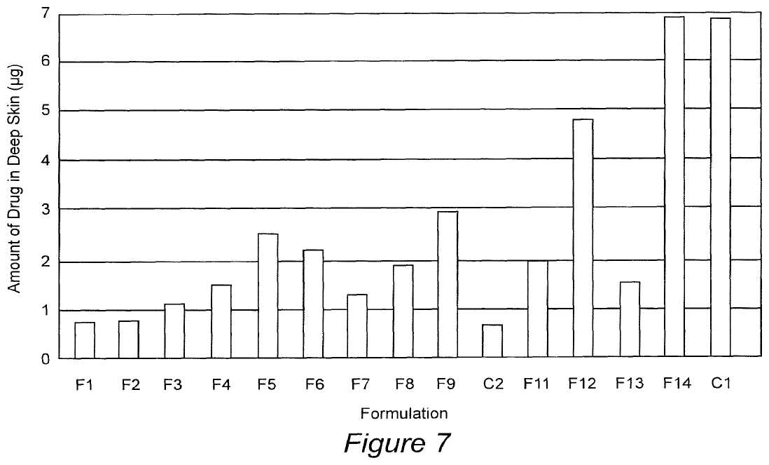

Formulations with 6% drug loading had better performance on the amount of drug permeated into deep skin than formulations with I % drug loading.
Formulations F14 and Control had the highest amount of drug permeated into deep skin.
   The amount of drug found in deep skin in first and second cadaver skin flux studies (Examples 8 and 11, respectively) is summarized in FIG. 7.

### Example 12. Formulation Chemical Stability Testing

Formulation F14 from Example 11 was tested for chemical stability as shown in Table 30. After the formulation was stored for 1 week at the temperature and humidity shown below, the amount of drug remaining was assayed by HPLC.

TABLE 30
Chemical stability of Formulation F14

| | wt % | % Remained based |
|---|---|---|
| Storage Condition | 25HC3S | on 1 month at 5° C. |
| 1 Month | 6.22 | — |
| 5° C. | (6.23, 6.21) | |
| 1 Month | 6.15 | 98.9 |
| 25° C./60% RH | 6.23 | 100.2 |
| 1 Month | 6.10 | 98.1 |
| 40° C./75% RH | 6.18 | 99.4 |

### Example 13. Formulation Physical and Chemical Stability Testing

### Methods

The formulations shown in below Tables 31 and 32 were made using the procedures described in Example 10.

### Results

The appearance of the resulting formulations is shown in below Tables 31 and 32. The 6 wt % 25HC3S was all in solution in the prepared compositions. The formulations of Table 32 were left at room temperature for one month, and their physical stability was recorded.

TABLE 31
Formulations for Physical Stability Studies

| Components, | | | | |
|---|---|---|---|---|
| % w/w | Form ID 57 | Form ID 58 | Form ID 59 | Form ID 60 |
| 25HC3S | 6 | 6 | 6 | 6 |
| PEG 400 | 9.4 | 4.8 | 4.7 | 4.8 |
| ETOH | 56.4 | 56.4 | 56.4 | 56.4 |
| DMSO | | 14.2 | 14.1 | 18.7 |
| Propylene | 21.6 | 12 | 14.1 | 9.4 |
| Glycol | | | | |
| Water | 1.9 | 1.9 | | |
| Silicon Dioxide | 4.7 | 4.7 | 4.7 | 4.7 |
| Appearance | Low | Low | Low | Low |
| | viscosity | viscosity | viscosity | viscosity |
| | opaque gel | opaque gel | opaque gel | opaque gel |
| Physical stability | NT | NT | NT | NT |
| at room | | | | |
| temperature after | | | | |
| 1 month | | | | |

NT: Not Tested

TABLE 32
Formulations for Physical Stability Studies

| Components, | Form | Form | Form | Form |
|---|---|---|---|---|
| % w/w | ID 61 | ID 62 | ID 63 | ID 64 |
| 25HC3S | 6 | 6 | 1 | 1 |
| PEG 400 | 5 | 5 | | — |
| ETOH | 10 | 56 | 79 | 78 |
| DMSO | — | 19 | 20 | 20 |
| Propylene Glycol | 60 | 8 | | — |
| Oleyl alcohol | 2 | — | | — |
| Oleic acid | 10 | 1 | | 1 |
| Water | 2 | — | | — |
| Silicon dioxide, SiO2 | 5 | 5 | | — |
| Appearance | Thin gel | gel | Slightly | Solution |
| | | | turbid | |
| | | | Solution | |
| Physical stability at | Stable | Phase | Stable | Stable |
| room temperature | | separated | | |
| | | after | | |
| | | 1 day | | |

Formulations 61 and 64 were tested for chemical stability as shown in Table 33. After the formulation was stored for 1 week at the temperature and humidity shown below, the amount of drug remaining was assayed by HPLC.

TABLE 33
Chemical Stability of Formulations after 1 week at 40° C./75% RH

| | | | % Remained |
|---|---|---|---|
| | | Theoretical | based |
| Formulation | Storage | Concentration, | Theoretical |
| ID | Condition | mg/g | Concentration |
| Form ID 61 | 1 week | 59.83 | 101.7 |
| Form ID 64 | 40° C./75% RH | 10.06 | 99.8 |

### Example 14. Treatment of Psoriasis (Prophetic)

### Objective

To investigate the efficacy of the active compound in patients with psoriasis vulgaris (i.e., plaque psoriasis).

### Formulation

The active compound, 25HC3S, is prepared in two formulations as shown in the below Table 34. The placebo contains the same excipients without the active compound.

TABLE 34

| Components, | | |
|---|---|---|
| % w/w | Form ID 61 | Form ID 64 |
| 25HC3S | 6 | 1 |
| PEG 400 | 5 | — |
| ETOH | 10 | 78 |
| DMSO | — | 20 |
| Propylene Glycol | 60 | — |
| Oleyl alcohol | 2 | — |
| Oleic acid | 10 | 1 |
| Water | 2 | — |
| Silicon dioxide, SiO2 | 5 | — |

### Methodology

This is a randomized, investigator-blinded, placebo-controlled, exploratory clinical study.

Male and female patients with mild, moderate to severe psoriasis vulgaris will be enrolled. Patients should discontinue all other treatments for psoriasis for at least a period of 4 weeks before study initiation (depending on the treatment they were on before). All patients receive simultaneous application of active and placebo formulations on symmetric plaques. A total of at least 10 patients per formulation are enrolled and treated.

In the trial, the active or placebo is applied daily to weekly to affected areas of the body for 1 to 4 weeks. The dose is 1 mg/cm² to 60 mg/cm². The treatment results are evaluated at weekly intervals until week 4 and then followed up for 1 to 12 months after discontinuation of the study medication.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

For all numeric ranges provided herein, it should be understood that the ranges include all integers between the highest and lowest value of the range, as well as all decimal fractions lying between those values, e.g. in increments of 0.1.

For all numeric values provided herein, the value is intended to encompass all statistically significant values surrounding the numeric value.

While the invention has been described in terms of its preferred embodiments, those skilled in the art will recognize that the invention can be practiced with modification within the spirit and scope of the appended aspects and claims. Accordingly, the present invention should not be limited to the embodiments as described above, but should further include all modifications and equivalents thereof within the spirit and scope of the description provided herein.

1-38. (canceled)

39. A composition comprising:
polyethylene glycol;
ethanol;
propylene glycol;
oleyl alcohol;
oleic acid; and
water.

40. The composition of claim 39, wherein the polyethylene glycol is present in the composition in an amount ranging from 1 wt % to 98 wt %, based on weight of the composition.

41. The composition of claim 39, wherein the polyethylene glycol is present in the composition in an amount ranging from 5 wt % to 50 wt %, based on weight of the composition.

42. The composition of claim 39, wherein the ethanol is present in the composition in an amount ranging from 1 wt % to 98 wt %, based on weight of the composition.

43. The composition of claim 39, wherein the ethanol is present in the composition in an amount ranging from 5 wt % to 50 wt %, based on weight of the composition.

44. The composition of claim 39, wherein the ethanol is present in the composition in an amount ranging from 7 wt % to 20 wt %, based on weight of the composition.

45. The composition of claim 39, wherein the propylene glycol is present in the composition in an amount ranging from 1 wt % to 98 wt %, based on weight of the composition.

46. The composition of claim 39, wherein the oleyl alcohol is present in the composition in an amount ranging from 1 wt % to 98 wt %, based on weight of the composition.

47. The composition of claim 39, wherein the oleic acid is present in the composition in an amount ranging from 1 wt % to 98 wt %, based on weight of the composition.

48. The composition of claim 39, wherein the oleic acid is present in the composition in an amount ranging from 5 wt % to 50 wt %, based on weight of the composition.

49. The composition of claim 39, wherein the oleic acid is present in the composition in an amount ranging from 7 wt % to 20 wt %, based on weight of the composition.

50. The composition of claim 39, wherein the water is present in the composition in an amount ranging from 0.5 wt % to 90 wt %, based on weight of the composition.

51. The composition of claim 39, wherein the water is present in the composition in an amount ranging from 1 wt % to 10 wt %, based on weight of the composition.

52. A method of administering a composition comprising:
topically administering a composition comprising polyethylene glycol, ethanol, propylene glycol, oleyl alcohol, oleic acid, and water.

53. The method of claim 52, wherein the polyethylene glycol is present in the composition in an amount ranging from 5 wt % to 50 wt %, based on weight of the composition.

54. The method of claim 52, wherein the ethanol is present in the composition in an amount ranging from 7 wt % to 20 wt %, based on weight of the composition.

55. The method of claim 52, wherein the propylene glycol is present in the composition in an amount ranging from 1 wt % to 98 wt %, based on weight of the composition.

56. The method of claim 52, wherein the oleyl alcohol is present in the composition in an amount ranging from 1 wt % to 98 wt %, based on weight of the composition.

57. The method of claim 52, wherein the oleic acid is present in the composition in an amount ranging from 7 wt % to 20 wt %, based on weight of the composition.

58. The method of claim 52, wherein the water is present in the composition in an amount ranging from 1 wt % to 10 wt %, based on weight of the composition.
